(19) United States
(12) Patent Application Publication
(10) Pub. No.: US 2023/0355527 A1
(43) Pub. Date: Nov. 9, 2023

(54) COMPOUNDS AND COMPOSITIONS FOR DRUG DELIVERY

(71) Applicant: RNAimmune, Inc., Germantown, MD (US)

(72) Inventors: Evan Michael Lewoczko, Gaithersburg, MD (US); Renxiang Chen, Gaithersburg, MD (US); Neeti Ananthaswamy, Gaithersburg, MD (US); Dong Shen, Gaithersburg, MD (US)

(73) Assignee: RNAimmune, Inc., Germantown, MD (US)

(21) Appl. No.: 18/297,255

(22) Filed: Apr. 7, 2023

Related U.S. Application Data
(60) Provisional application No. 63/476,135, filed on Dec. 19, 2022.
(60) Provisional application No. 63/476,131, filed on Dec. 19, 2022.
(60) Provisional application No. 63/328,367, filed on Apr. 7, 2022.

(51) Int. Cl. A61K 9/127; C07C 233/47; C07C 229/14; C07C 323/30; C07C 327/22; C07C 323/60; C07C 229/12; C07C 229/16; C07C 239/16; C07C 237/06; C07C 271/20; C07C 237/12; C07C 309/14; C07C 239/18

(52) U.S. Cl. CPC A61K 9/1272; C07C 229/12; C07C 229/14; C07C 229/16; C07C 233/47; C07C 237/06; C07C 237/12; C07C 239/16; C07C 239/18; C07C 271/20; C07C 309/14; C07C 323/30; C07C 323/60; C07C 327/22; C07C 2601/04

(57) ABSTRACT

Novel lipids, compositions, and methods of using the novel lipids and compositions are disclosed. Three-component lipid nanoparticle compositions comprising the novel lipids or other types of lipids, and methods of using the three-component lipid nanoparticle compositions are disclosed. Three-component lipid nanoparticle compositions contain a steroidal or structural lipid-containing component, a PEGylated lipid-containing component, a cationic or ionizable lipid-containing component, and are free of phospholipids. Pharmaceutical formulations including the three-component lipid nanoparticle compositions and further including therapeutic and/or prophylactics such as mRNA are useful in the delivery of therapeutic and/or prophylactics to mammalian cells or organs.

### CROSS-REFERENCE TO RELATED APPLICATIONS

This application claims the benefit of U.S. Provisional Application No. 63/328,367, filed Apr. 7, 2022, U.S. Provisional Application No. 63/476,131, filed Dec. 19, 2022, and U.S. Provisional Application No. 63/476,135, filed Dec. 19, 2022. The entire contents of the above-identified applications are hereby fully incorporated herein by reference.

### TECHNICAL FIELD

The present disclosure provides novel lipid nanoparticle compositions and methods to deliver one or more therapeutic and/or prophylactics to and/or produce polypeptides in mammalian cells or organs.

### BACKGROUND

Delivery of biologically active substances such as small molecule drugs, proteins, and nucleic acids including mRNA is a medical challenge. In particular, the delivery of nucleic acids to cells is made difficult by the relative instability and low cell permeability of such molecules. Currently approved lipid nanoparticle (LNP) compositions require a mixture of four components: phospholipid(s); cholesterol; PEGylated lipid(s); and cationic or ionizable lipid(s), e.g., for delivery mRNA vaccines. The phospholipids and cholesterol are used to provide the necessary structure and stability, the PEGylated lipids support prolonged circulation, and the cationic/ionizable lipids are for complexing of the negatively charged mRNA molecules and enable the exit of the mRNA from the endosome to the cytosol for translation. There exists a need to develop compositions and methods for improved delivery of therapeutic and/or prophylactics molecules into cells or organs.

### SUMMARY OF THE INVENTION

The present disclosure provides novel compositions and methods involving LNPs formed from a mixture of three components.

In one aspect, the present disclosure provides a three-component LNP composition wherein the three components are:
1) a steroidal or structural lipid-containing component;
2) a PEGylated lipid-containing component; and
3) a cationic or ionizable lipid-containing component.

Thus, the three-component LNP composition of the present disclosure does not contain a phospholipid-containing component.

In one aspect, the three-component LNP composition contains the three components in the following relative mole percentages:
1) 5 to 60 mole% of a steroidal or structural lipid-containing component;
2) 0.5 to 20 mole% of a PEGylated lipid-containing component; and
3) 30 to 70 mole% of a cationic or ionizable lipid-containing component.

In one aspect, the three-component LNP composition contains the three components in the following relative mole percentages:
1) 20 to 50 mole% of a steroidal or structural lipid-containing component;
2) 0.8 to 10 mole% of a PEGylated lipid-containing component; and
3) 40 to 62 mole% of a cationic or ionizable lipid-containing component.

In one aspect, the three-component LNP composition contains the three components in the following relative mole percentages:
1) 25 to 46 mole% of a steroidal or structural lipid-containing component;
2) 1 to 7 mole% of a PEGylated lipid-containing component; and
3) 44 to 58 mole% of a cationic or ionizable lipid-containing component.

In one aspect, the three-component LNP composition contains the three components in the following relative mole percentages:
1) 35 to 44 mole% of a steroidal or structural lipid-containing component;
2) 1.2 to 5 mole% of a PEGylated lipid-containing component; and
3) 48 to 57 mole% of a cationic or ionizable lipid-containing component.

In one aspect, the cationic or ionizable lipid-containing component may comprise MC3, ALC-0315, ALC-0159, SM-102, 1,2-dioleoyl-3-trimethylammonium propane (DOTAP), Mol-111, Mol-114, MH-094, or a cationic and/or ionizable lipid disclosed in WO2017049245A2 (Benenato), which is incorporated herein by reference.

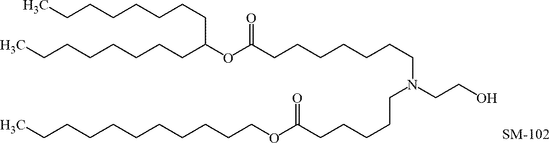

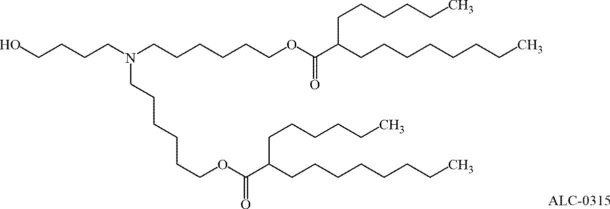

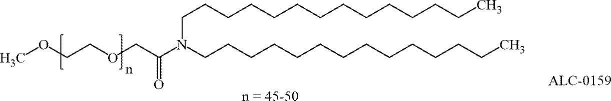

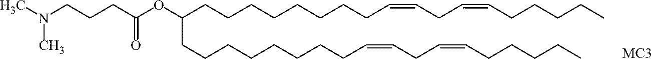

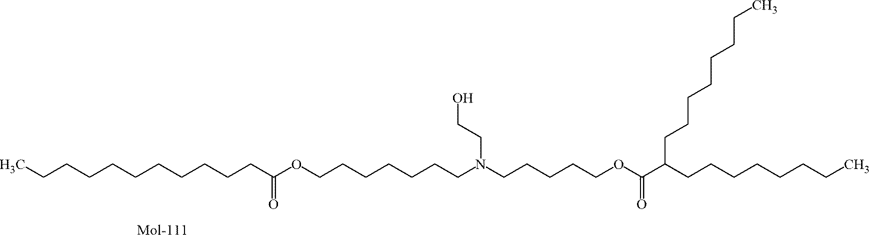

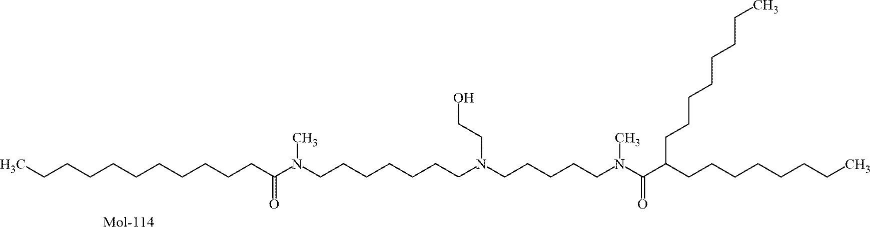

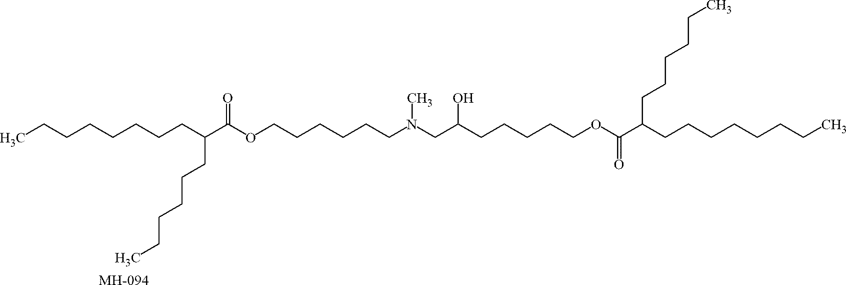

In one aspect, the cationic or ionizable lipid-containing component may comprise compounds of Formula (IA):

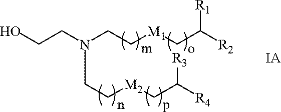

or a salt or isomer thereof, wherein
m is 0-9;
n is 0-9;
o is 0-12;
p is 0-12;
R₁ is a linear C₁₋₁₂ alkyl;
R₂ is H or a linear C₁₋₁₂ alkyl;
R₃ is a linear C₁₋₁₂ alkyl;
R₄ is H or linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IA may include, for example, the following compounds:

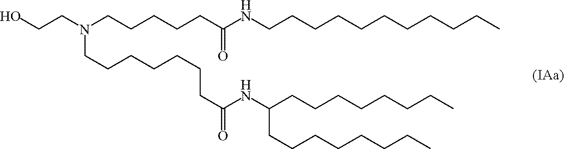 [SMILES: CCCCCCCCCCCNC(=O)CCCCCN(CCO)CCCCCCCC(=O)NC(CCCCCCCC)CCCCCCCC]

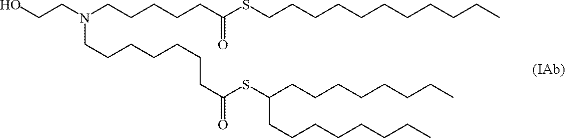 [SMILES: CCCCCCCCCCCSC(=O)CCCCCN(CCO)CCCCCCCC(=O)SC(CCCCCCCC)CCCCCCCC]

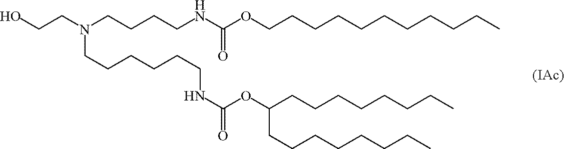 [SMILES: CCCCCCCCCCCOC(=O)NCCCCN(CCO)CCCCCCNC(=O)OC(CCCCCCCC)CCCCCCCC]

In another aspect, the present disclosure provides compounds of Formula (IB):

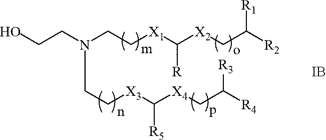

or a salt or isomer thereof, wherein
m is selected from 0-9;
n is selected from 0-9;
o is selected from 0-12;
p is selected from 0-12;
R is the side chain of an independently selected amino acid;
R₁ is a linear C₁₋₁₂ alkyl;
R₂ is H or linear C₁₋₁₂ alkyl;
R₃ is a linear C₁₋₁₂ alkyl;
R₄ is H or linear C₁₋₁₂ alkyl;
R₅ is the side chain of an independently selected amino acid;
X₁ is —OC(O)N(H)—, —C(O)N(H)—, —N(H)C(O)—, or —OC(O)—;
X₂ is —C(O)N(H)—, —C(O)O—, —N(H)C(O)—, or —N(H)C(O)—;
X₃ is —OC(O)N(H)—, —C(O)N(H)—, —N(H)C(O)—, or —OC(O)—; and
X₄ is —C(O)N(H)—, —C(O)O—, —N(H)C(O)—, or —N(H)C(O)—.

In some aspects, R or R₅ comprises the side chain of a Serine (S), Threonine (T), Cysteine (C), Selenocysteine (U), Glycine (G), Alanine (A), Isoleucine (I), Leucine (L), Methionine (M), or Valine (V). In some aspects, the carbonyl group in Formula IB is bonded to the amino terminus of the amino acid. In some aspects, the carbonyl group in Formula IB is bonded to the carboxy terminus of the amino acid.

In certain aspects, compounds of Formula IB may include, for example, the following compounds:

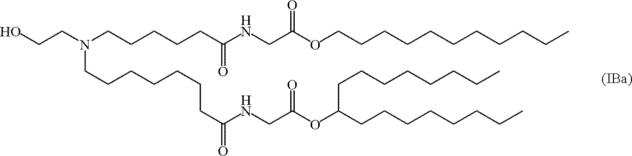 [SMILES: CCCCCCCCCCCOC(=O)CNC(=O)CCCCCN(CCO)CCCCCCCC(=O)NCC(=O)OC(CCCCCCCC)CCCCCCCC]

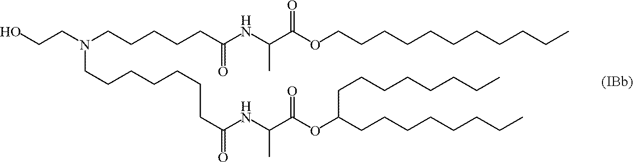 [SMILES: CCCCCCCCCCCOC(=O)C(C)NC(=O)CCCCCN(CCO)CCCCCCCC(=O)NC(C)C(=O)OC(CCCCCCCC)CCCCCCCC]

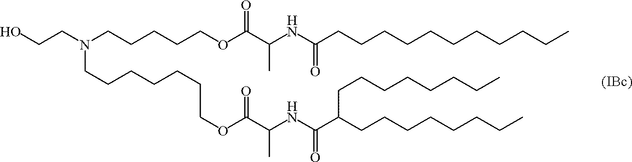 [SMILES: CCCCCCCCCCCC(=O)NC(C)C(=O)OCCCCCN(CCO)CCCCCCCOC(=O)C(C)NC(=O)C(CCCCCCCC)CCCCCCCC]

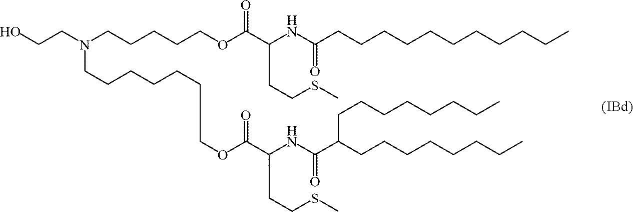 [SMILES: CCCCCCCCCCCC(=O)NC(CCSC)C(=O)OCCCCCN(CCO)CCCCCCCOC(=O)C(CCSC)NC(=O)C(CCCCCCCC)CCCCCCCC]

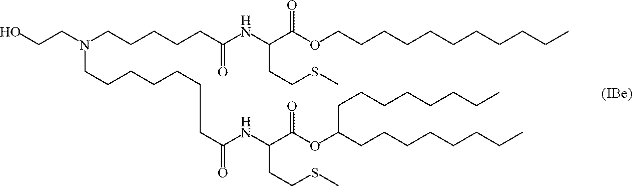 [SMILES: CCCCCCCCCCCOC(=O)C(CCSC)NC(=O)CCCCCN(CCO)CCCCCCCC(=O)NC(CCSC)C(=O)OC(CCCCCCCC)CCCCCCCC]

In another aspect, the present disclosure provides compounds of Formula (IC):

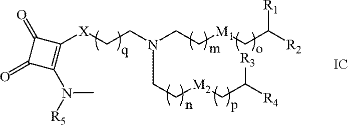

or a salt or isomer thereof, wherein
m is selected from 0-9;
n is selected from 0-9;
o is selected from 0-12;
p is selected from 0-12;
q is selected from 0-5;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl;
R₅ is H or CH₃;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; and
X is selected from —CH₂—, —O—, —S—, or —P(O)(OR)O—.

In certain aspects, compounds of Formula IC may include, for example, the following compounds:

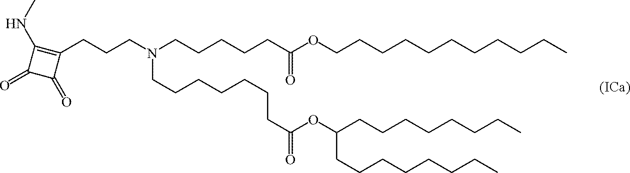 [SMILES: CCCCCCCCCCCOC(=O)CCCCCN(CCCCCCCC(=O)OC(CCCCCCCC)CCCCCCCC)CCCc1c(NC)c(=O)c1=O]

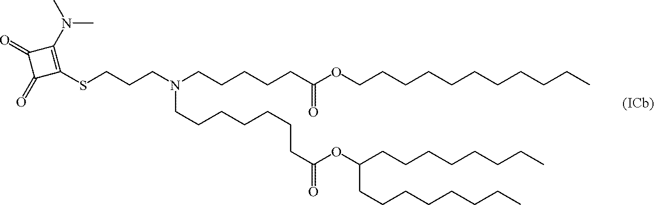 [SMILES: CCCCCCCCCCCOC(=O)CCCCCN(CCCCCCCC(=O)OC(CCCCCCCC)CCCCCCCC)CCCSc1c(N(C)C)c(=O)c1=O]

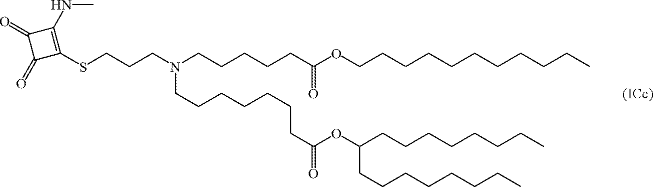 [SMILES: CCCCCCCCCCCOC(=O)CCCCCN(CCCCCCCC(=O)OC(CCCCCCCC)CCCCCCCC)CCCSc1c(NC)c(=O)c1=O]

In one aspect, the cationic or ionizable lipid-containing component may comprise compounds of Formula (IIA):

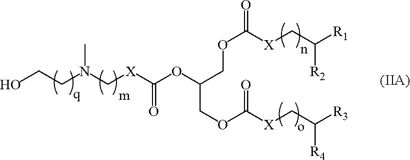

or a salt or isomer thereof, wherein
m is selected from 0-5;
n is selected from 0-12;
o is selected from 0-12;
q is selected from 1-3;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
X is selected from C(R)₂, N(R), or O, wherein R is independently selected from a methyl and H.

In certain aspects, compounds of Formula IIA may include, for example, the following compound.

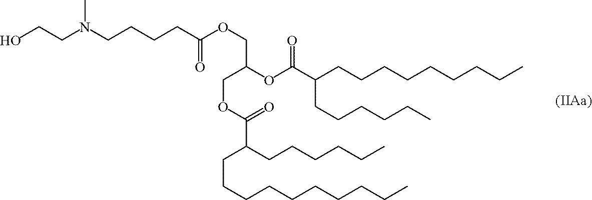 [SMILES: CCCCCCCCCCC(CCCCCC)C(=O)OCC(COC(=O)CCCCN(C)CCO)OC(=O)C(CCCCCC)CCCCCCCCC]

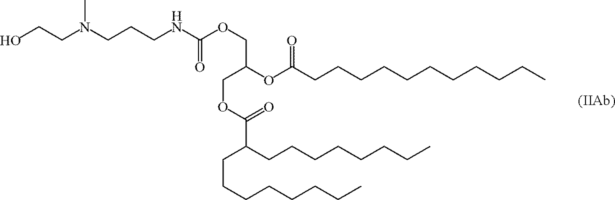 [SMILES: CCCCCCCCCCCC(=O)OC(COC(=O)NCCCN(C)CCO)COC(=O)C(CCCCCCCC)CCCCCCCC]

In another aspect, the present disclosure provides compounds of Formula (IIB):

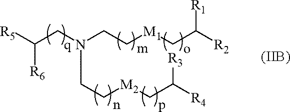

or a salt or isomer thereof, wherein
m is selected from 0-9;
n is selected from 0-9;
o is selected from 0-12;
p is selected from 0-12;
q is selected from 0-6;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IIB may include, for example

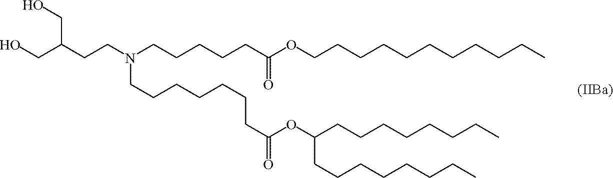 [SMILES: CCCCCCCCCCCOC(=O)CCCCCN(CCCCCCCC(=O)OC(CCCCCCCC)CCCCCCCC)CCC(CO)CO]

In another aspect, the present disclosure provides compounds of Formula (IIC):

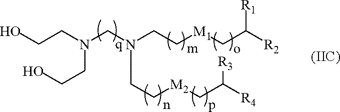

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IIC may include, for example

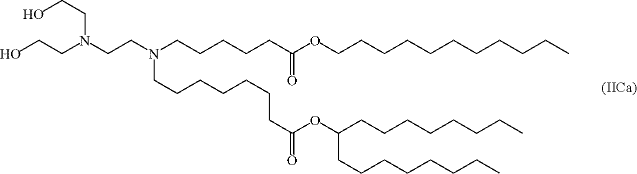 [SMILES: CCCCCCCCCCCOC(=O)CCCCCN(CCCCCCCC(=O)OC(CCCCCCCC)CCCCCCCC)CCN(CCO)CCO]

In another aspect, the present disclosure provides compounds of Formula (IID):

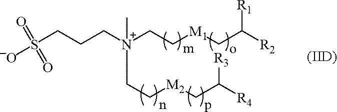

or a salt or isomer thereof, wherein
m is selected from 0-9;
n is selected from 1-7;
o is selected from 0-12;
p is selected from 0-12;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IID may include, for example

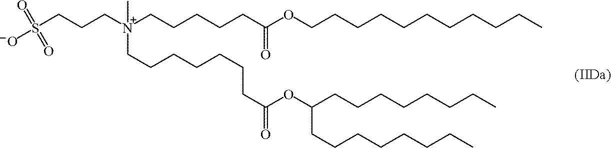 [SMILES: CCCCCCCCCCCOC(=O)CCCCC[N+](C)(CCCCCCCC(=O)OC(CCCCCCCC)CCCCCCCC)CCCS(=O)(=O)[O-]]

In another aspect, the present disclosure provides compounds of Formula (IIE):

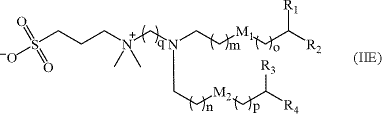

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IIE may include, for example

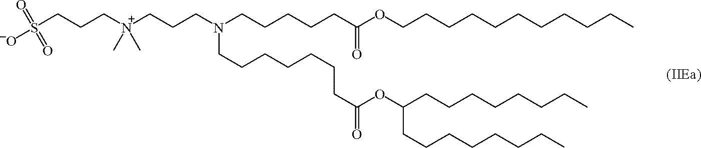 [SMILES: CCCCCCCCCCCOC(=O)CCCCCN(CCCCCCCC(=O)OC(CCCCCCCC)CCCCCCCC)CCC[N+](C)(C)CCCS(=O)(=O)[O-]]

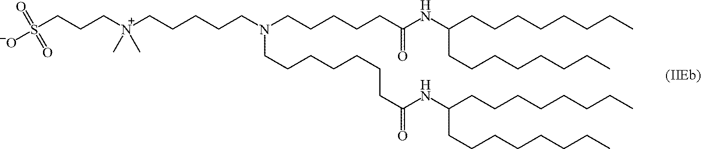 [SMILES: CCCCCCCCC(CCCCCCCC)NC(=O)CCCCCCCN(CCCCCC(=O)NC(CCCCCCCC)CCCCCCCC)CCCCC[N+](C)(C)CCCS(=O)(=O)[O-]]

In another aspect, the present disclosure provides compounds of Formula (IIF):

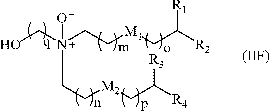

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In certain aspects, compounds of Formula IIF may include, for example

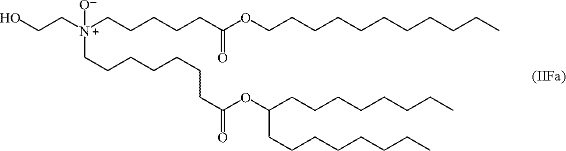 [SMILES: CCCCCCCCCCCOC(=O)CCCCC[N+]([O-])(CCO)CCCCCCCC(=O)OC(CCCCCCCC)CCCCCCCC]

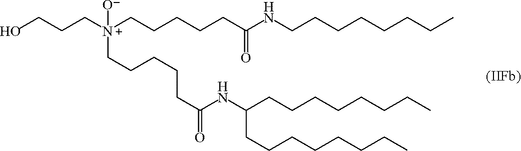 [SMILES: CCCCCCCCNC(=O)CCCCC[N+]([O-])(CCCO)CCCCCC(=O)NC(CCCCCCCC)CCCCCCCC]

In another aspect, the present disclosure provides a method of delivering a therapeutic and/or prophylactic (e.g., an mRNA) to a cell (e.g., a mammalian cell) by administering a three-component LNP composition containing the steroidal or structural lipid-containing component, the PEGylated lipid-containing component, and the cationic or ionizable lipid-containing component, to deliver the therapeutic and/or prophylactic to a subject (e.g., a mammal, such as a human), in which administering involves contacting the cell with the three-component LNP composition such that the therapeutic and/or prophylactic is delivered to the cell.

In another aspect, the present disclosure provides a method of producing a polypeptide of interest in a cell (e.g., a mammalian cell) by contacting the cell with the three-component LNP composition and an mRNA encoding the polypeptide of interest, whereby the mRNA is capable of being translated in the cell to produce the polypeptide.

### BRIEF DESCRIPTION OF THE DRAWINGS

FIG. 1 shows a Western blot and data of in vitro expression of protein from mRNA encapsulated in the three-component LNP composition of the present disclosure and four-component LNP (comparator) composition (“RL007”). The top image is raw data and the bottom graph is processed data by ImageJ.

FIG. 2 is the ELISA results of animal study. Briefly, plates were coated with Covid-19 delta S1 (from Sino Biological) and delta RBD (from eEnzyme) proteins, respectively. Then the sera from immunized mice were added to the plates coated with delta S1 and delta RBD. The results showed the amount of antibody that can bind to antigen for mRNA encapsulated in the three-component LNP composition of the present disclosure (right) and four-component LNP (comparator) (left) composition in mice.

FIG. 3 shows encapsulation data for 3-component and 4-component LNPs. One through four (1-4) are the encapsulation results for 3-component LNPs prepared at pH 4.0 with different concentrations of lipids and different PEGylated lipids. Five (5) is the encapsulation result for the control 4-component LNP (“RL007”). Six (6) and seven (7) are LNPs prepared at pH 6.0. Six (6) is the control 4-component LNP (RL007) and seven (7) is the 3-component LNP that was also used in the in vivo study (FIG. 2).

FIG. 4 shows in vivo expression of a series of mLNPs compared to a 4-component control LNP.

FIG. 5 shows in vitro expression comparison of 4-component control LNPs with two ionizable lipids (SM-102 and Mol-111) with the same mRNA and relative lipid concentrations (SM-102_LNP and Mol-111_LNP) to 3-component LNPs with the same two ionizable lipids (SM-102 and Mol-111), the same mRNA and relative lipid concentrations as the LNPs (SM-102_mLNP and Mol-111_mLNP).

FIGS. 6A-6B show in vivo expression across serial dilution demonstrating compatibility of mLNP with mRNA of different lengths and multiple types of ionizable lipids. FIG. 6A shows results for LNPs that are either empty (SM-102 LNP (blank)) or contain Covid Delta Spike mRNA (~4000 nb); FIG. 6B shows results for LNPs that contain RSV mRNA (~2000 nb).

FIG. 7 shows results that demonstrate the difference in pKa between LNP (far left) and mLNP plots.

FIG. 8 show stability of mLNPs compared to a control LNP.

### DETAILED DESCRIPTION

The disclosure relates to novel lipids and novel three-component LNP composition compositions. The disclosure also provides methods of delivering a therapeutic and/or prophylactic to a mammalian cell, specifically delivering a therapeutic and/or prophylactic to a mammalian organ, producing a polypeptide of interest in a mammalian cell, and treating a disease or disorder in a mammal in need thereof. For example, a method of producing a polypeptide of interest in a cell involves contacting a three-component LNP composition comprising an mRNA with a mammalian cell, whereby the mRNA may be translated to produce the polypeptide of interest. A method of delivering a therapeutic and/or prophylactic to a mammalian cell or organ may involve administration of a three-component LNP composition including the therapeutic and/or prophylactic to a subject, in which the administration involves contacting the cell or organ with the three-component LNP composition, whereby the therapeutic and/or prophylactic is delivered to the cell or organ.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

Alkyl, alkenyl, and cyclyl (e.g., carbocyclyl and heterocyclyl) groups may be optionally substituted unless otherwise specified.

As used herein, the terms “approximately” and “about,” as applied to one or more values of interest, refer to a value that is similar to a stated reference value. In certain embodiments, the term “approximately” or “about” may refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “compound,” is meant to include all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, “expression” of a nucleic acid sequence refers to translation of an mRNA into a polypeptide or protein and/or post-translational modification of a polypeptide or protein.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (-)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

As used herein, a “lipid component” is that component of a nanoparticle composition that includes one or more lipids. For example, the lipid component may include one or more cationic/ionizable, PEGylated, or steroidal/structural lipid.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a nanoparticle composition including a lipid component and an RNA.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin D, vitamin E (alpha-tocopherol), vitamin C, vitamin K, xylitol, and other species disclosed herein.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington’s Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell. In certain aspects, the three-component LNP of the present disclosure is free of phospholipids, i.e., does not have the phospholipid component used in the traditional four-component LNP compositions.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, or a mixture thereof.

As used herein, “size” or “mean size” in the context of nanoparticle compositions refers to the mean diameter of a nanoparticle composition.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

### Novel Lipids

The present disclosure discloses novel lipids and lipid nanoparticle compositions comprising such novel lipids.

In one aspect, the present disclosure provides compounds of Formula IA:

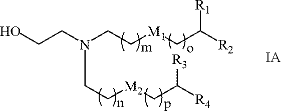

or a salt or isomer thereof, wherein
m is 0-9;
n is 0-9;
o is 0-12;
p is 0-12;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups wherein R is a H or a methyl group. Synthesis Scheme 1. General synthesis route for the synthesis of compounds of Formula IA.

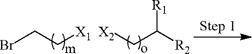

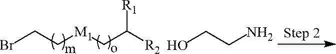

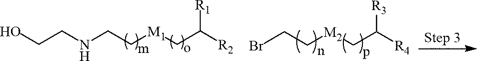

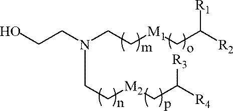

Scheme 2. Fatty acid tail synthesis of R₁ and R₂, which also applies to synthesis of R₃ and R₄.

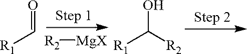

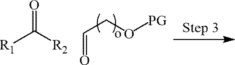

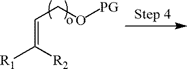

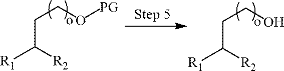

Scheme 3a. Fatty Acid tail conversion from hydroxy to thiol

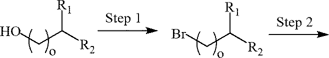

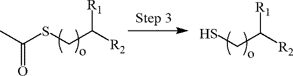

Scheme 3b. Fatty acid tail conversion of hydroxy to amine.

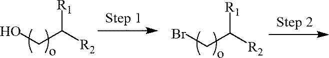

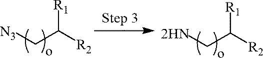

Scheme 4. Fatty acid tail conversion of hydroxy to carboxylic acid.

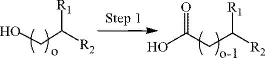

Scheme 5. Fatty acid tail conversion of hydroxy to isocyanate.

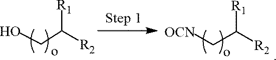

In certain aspects, compounds of Formula IA may include, for example, the following compounds:

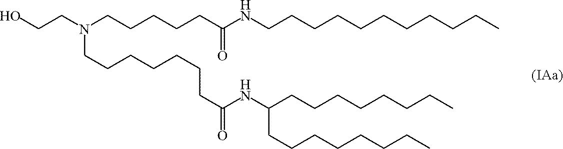 [SMILES: CCCCCCCCCCCNC(=O)CCCCCN(CCO)CCCCCCCC(=O)NC(CCCCCCCC)CCCCCCCC]

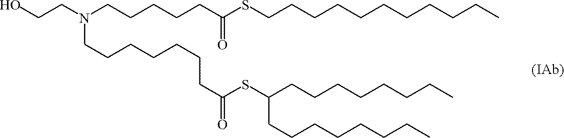 [SMILES: CCCCCCCCCCCSC(=O)CCCCCN(CCO)CCCCCCCC(=O)SC(CCCCCCCC)CCCCCCCC]

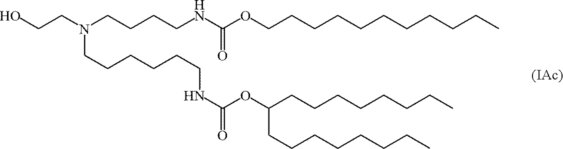 [SMILES: CCCCCCCCCCCOC(=O)NCCCCN(CCO)CCCCCCNC(=O)OC(CCCCCCCC)CCCCCCCC]

In another aspect, the present disclosure provides compounds of Formula IB:

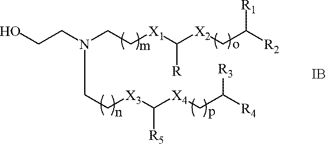

or a salt or isomer thereof, wherein
m is selected from 0-9;
n is selected from 0-9;
o is selected from 0-12;
p is selected from 0-12;
R is the side chain of an independently selected amino acid;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is the side chain of an independently selected amino acid;
X₁ is —OC(O)N(H)—, —C(O)N(H)—, —N(H)C(O)—, or —OC(O)—;
X₂ is —C(O)N(H)—, —C(O)O—, —N(H)C(O)—, or —N(H)C(O)—;
X₃ is —OC(O)N(H)—, —C(O)N(H)—, —N(H)C(O)—, or —OC(O)—;
X₄ is —C(O)N(H)—, —C(O)O—, —N(H)C(O)—, or —N(H)C(O)—. In some aspects, R or R₅ comprises the side chain of the amino acid, wherein the amino acid is Serine (S), Threonine (T), Cysteine (C), Selenocysteine (U), Glycine (G), Alanine (A), Isoleucine (I), Leucine (L), Methionine (M), or Valine (V).

In some aspects, the carbonyl group in Formula IB is bonded to the amino terminus of the amino acid. For example, a compound of Formula IB may have the following structure:

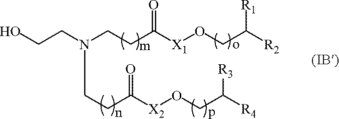

in which X₁ and X₂ represent independently an amino acid, wherein the amino acid is Serine (S), Threonine (T), Cysteine (C), Selenocysteine (U), Glycine (G), Alanine (A), Isoleucine (I), Leucine (L), Methionine (M), or Valine (V).

In some aspects, the carbonyl group in Formula IB is bonded to the carboxy terminus of the amino acid.

Synthesis Scheme 6. General synthetic route for the synthesis of compounds of Formula IB. Scheme 6a. General synthesis for the protection of the amino acid and orientation of the amino acid with the carbonyl group on the fatty acid tail side.

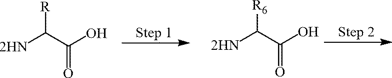

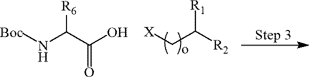

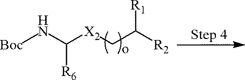

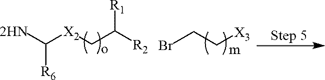

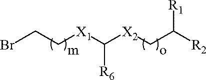

R is the selected amino acid side chain;
R₆ is the protected amino acid side chain;
X is a compatible functional group with carboxylic acid;
X₁ is N(H)C(O) or N(H)C(O);
X₂ is C(O)N(H) or C(O)O and;
X₃ is a compatible functional group with the amine.

Scheme 6b. General synthesis for the orientation of the amino acid so that the carbonyl is on the ionizable head group side of the lipid.

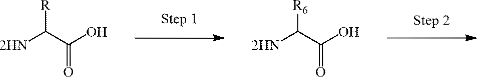

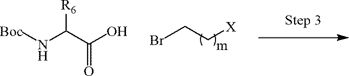

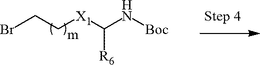

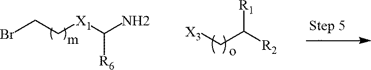

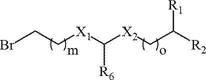

R is the selected amino acid side chain;
R₆ is the protected amino acid side chain;
X is a compatible functional group with carboxylic acid;
X₁ is C(O)N(H) or C(O)O;
X₂ is N(H)C(O) or N(H)C(O) and;
X₃ is a compatible functional group with the amine.

Scheme 6c. General synthetic scheme of the lipid with included amino acids.

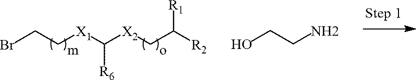

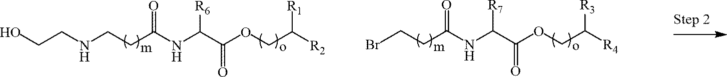

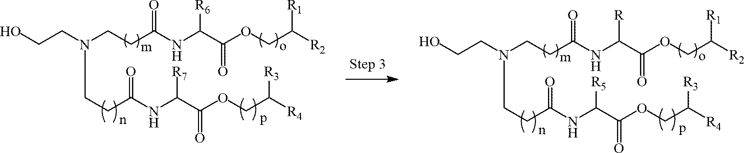

R₅ is the side chain of an independently selected amino acid side chain and;
R₇ is the protected side chain of an independently selected amino acid side chain. Synthesis of fatty acid tails are shown in Schemes 2 through 5 above.

In certain aspects, compounds of Formula IB may include, for example, the following compounds:

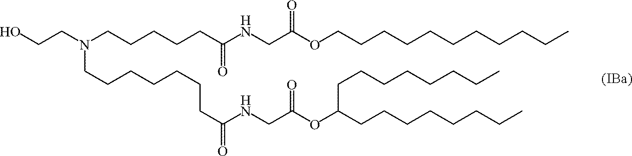 [SMILES: CCCCCCCCCCCOC(=O)CNC(=O)CCCCCN(CCO)CCCCCCCC(=O)NCC(=O)OC(CCCCCCCC)CCCCCCCC]

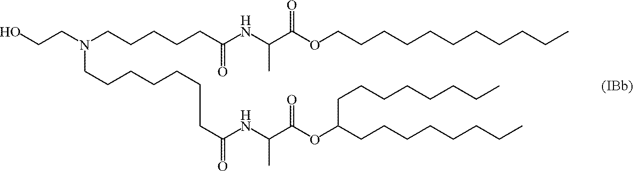 [SMILES: CCCCCCCCCCCOC(=O)C(C)NC(=O)CCCCCN(CCO)CCCCCCCC(=O)NC(C)C(=O)OC(CCCCCCCC)CCCCCCCC]

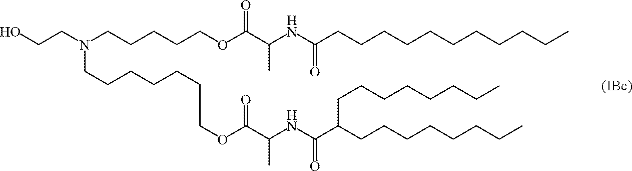 [SMILES: CCCCCCCCCCCC(=O)NC(C)C(=O)OCCCCCN(CCO)CCCCCCCOC(=O)C(C)NC(=O)C(CCCCCCCC)CCCCCCCC]

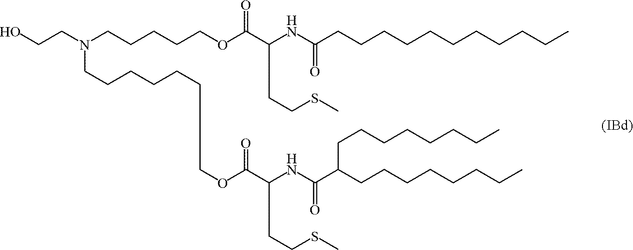 [SMILES: CCCCCCCCCCCC(=O)NC(CCSC)C(=O)OCCCCCN(CCO)CCCCCCCOC(=O)C(CCSC)NC(=O)C(CCCCCCCC)CCCCCCCC]

In another aspect, the present disclosure provides compounds of Formula IC:

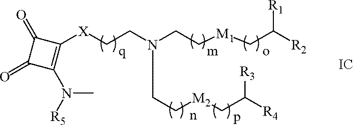

or a salt or isomer thereof, wherein
m is selected from 0-9;
n is selected from 0-9;
o is selected from 0-12;
p is selected from 0-12;
q is selected from 0-5;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is H or CH₃;
M₁ and M₂ are independently selected from —C(O)O—, —OC(O)—, —C(O)N(R)—, —N(R)C(O)—, —C(O)S—, —SC(O)—, —OC(O)O—, —OC(O)N(R)—, or —N(R)C(O)O— groups, wherein R is a H or a methyl group; and X is selected from —CH₂—, —O—, —S—, or —P(O)(OR)O—.

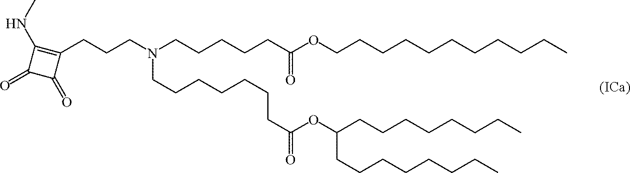 [SMILES: CCCCCCCCCCCOC(=O)CCCCCN(CCCCCCCC(=O)OC(CCCCCCCC)CCCCCCCC)CCCc1c(NC)c(=O)c1=O]

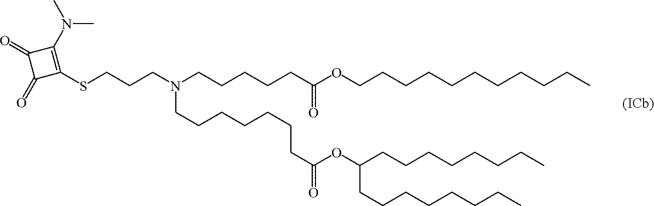 [SMILES: CCCCCCCCCCCOC(=O)CCCCCN(CCCCCCCC(=O)OC(CCCCCCCC)CCCCCCCC)CCCSc1c(N(C)C)c(=O)c1=O]

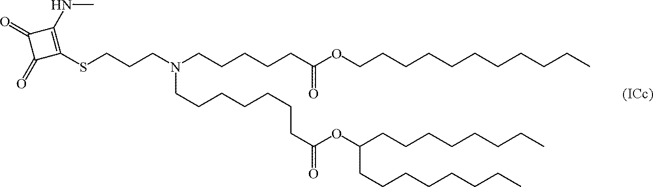 [SMILES: CCCCCCCCCCCOC(=O)CCCCCN(CCCCCCCC(=O)OC(CCCCCCCC)CCCCCCCC)CCCSc1c(NC)c(=O)c1=O]

Scheme 7. General synthetic route for the precursor to compounds of Formula IC.

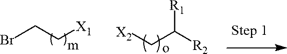

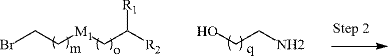

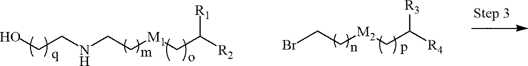

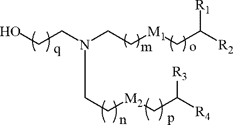

Scheme 8. Synthetic route of the conversion of the precursor hydroxy to thiol group.

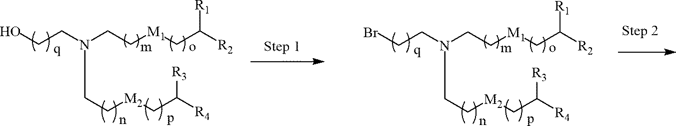

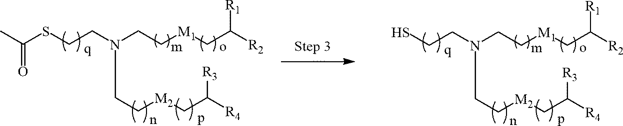

Scheme 9. Synthetic route of the conversion of the precursor hydroxy to amine group.

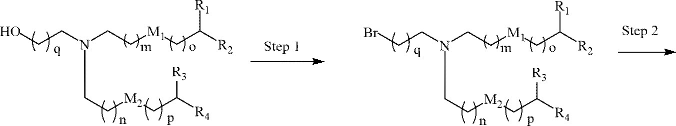

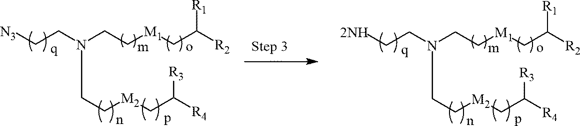

Scheme 10. Synthetic route of the conversion of the precursor hydroxy to phosphine group.

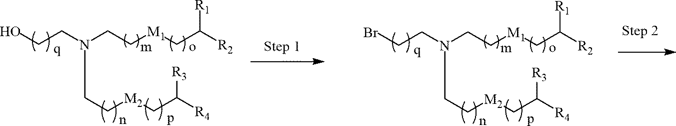

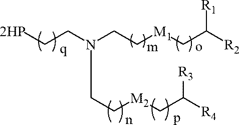

Scheme 11. General synthetic route of the synthesis of compound IC.

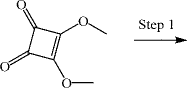

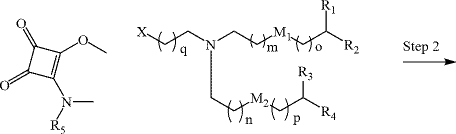

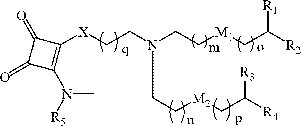

In another aspect, the present disclosure provides compounds of Formula IIA:

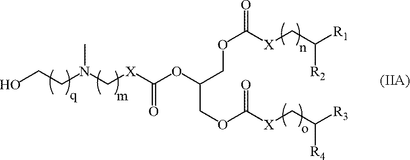

or a salt or isomer thereof, wherein
m is selected from 0-5;
n is selected from 0-12;
o is selected from 0-12;
q is selected from 1-3;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
X is selected from C(R)₂, N(R), or O, wherein R is independently selected from a methyl and H.

Scheme 12. General synthesis route for the synthesis of compounds of Formula IIA.

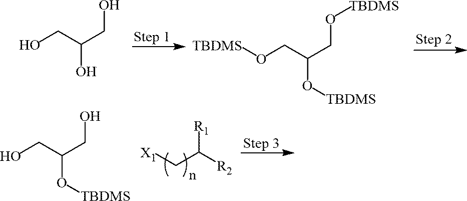

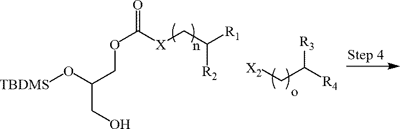

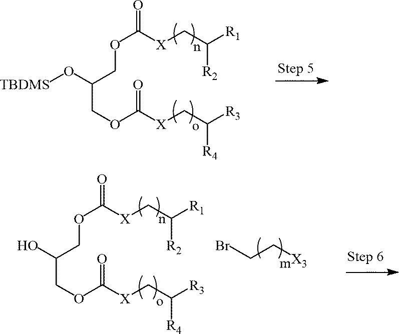

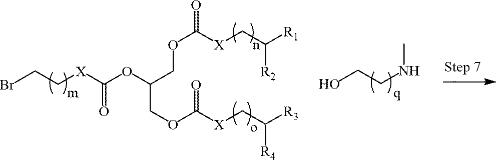

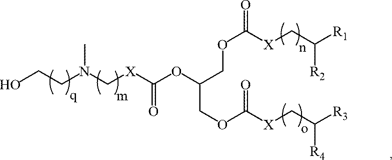

wherein X₁, X₂, and X₃ are either carboxylic acid (RC(O)O) functional groups, or they are isocyanate (RNCO) functional groups, and X is as defined above.

Scheme 13. Fatty acid tail synthesis for R₁ and R₂. The route is the same for R₃ and R₄.

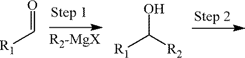

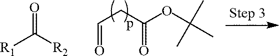

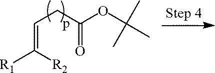

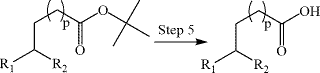

In another aspect, the present disclosure provides compounds of Formula IIB:

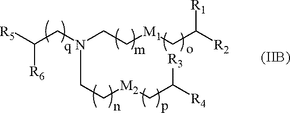

or a salt or isomer thereof, wherein
m is selected from 0-9;
n is selected from 0-9;
o is selected from 0-12;
p is selected from 0-12;
q is selected from 0-6;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

Scheme 14. General route for the synthesis of head groups of compounds of Formula IIB.

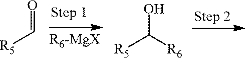

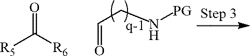

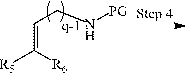

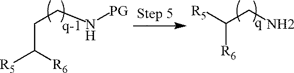

X is a halide and;
PG is a protecting group such as N-tert-butyloxycarbonyl group.

Scheme 15. General synthesis route for the compound IIB.

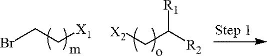

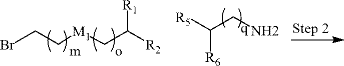

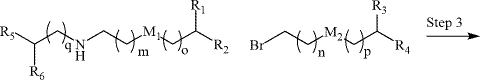

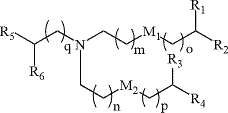

Scheme 16. Fatty Acid tail conversion from hydroxy to thiol

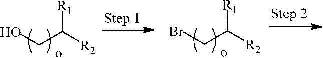

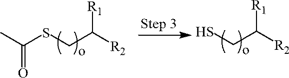

Scheme 17. Fatty acid tail conversion of hydroxy to amine.

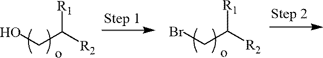

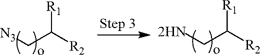

Scheme 18. Fatty acid tail conversion of hydroxy to carboxylic acid.

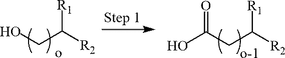

Scheme 19. Fatty acid tail conversion of hydroxy to isocyanate.

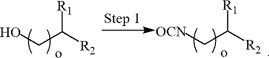

In another aspect, the present disclosure provides compounds of Formula IIC:

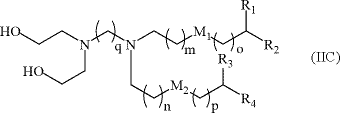

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

Scheme 20. General synthesis for compound IIC.

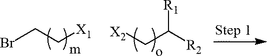

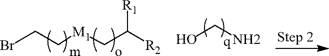

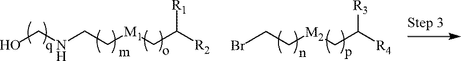

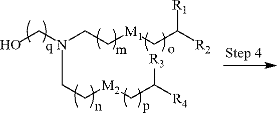

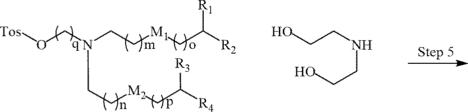

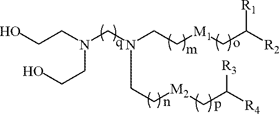

The side chains are synthesized according to Schemes 16-19.

In another aspect, the present disclosure provides compounds of Formula IID:

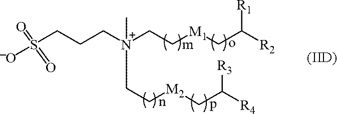

or a salt or isomer thereof, wherein
m is selected from 0-9;
n is selected from 1-7;
o is selected from 0-12;
p is selected from 0-12;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

Scheme 21. General route for the synthesis of the head groups for the synthesis of compounds of Formula IID.

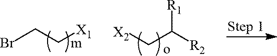

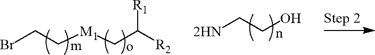

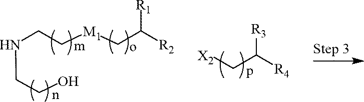

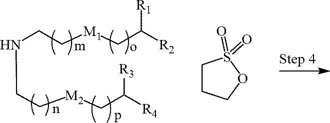

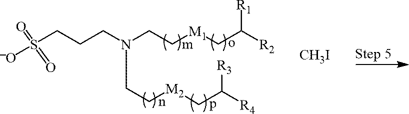

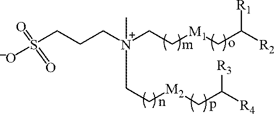

X₁ is a functional group (such as an amine, carboxylic acid, isocyante, etc) compatible with X₂ (an amine, carboxylic acid, isocyanate, etc) to give M₁ such that it is —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

In another aspect, the present disclosure provides compounds of Formula IIE:

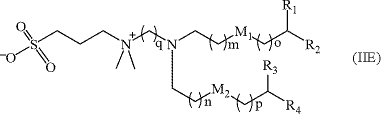

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

Scheme 21. General route for the synthesis of compound IIE.

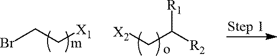

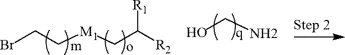

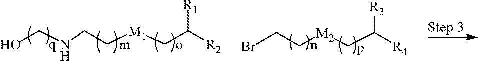

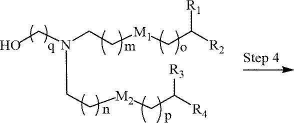

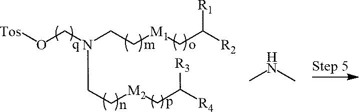

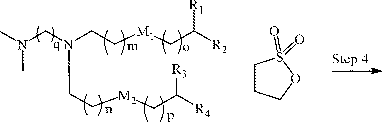

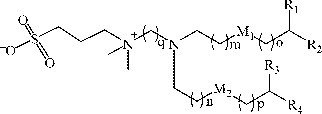

In another aspect, the present disclosure provides compounds of Formula IIF:

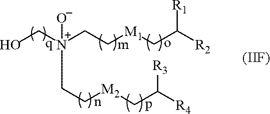

or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

Scheme 23. General route for the synthesis of compound IIF.

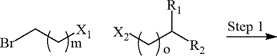

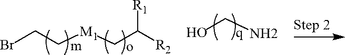

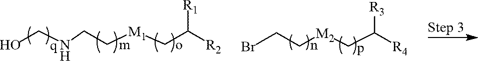

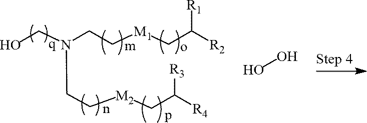

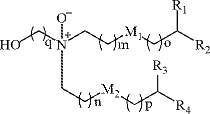

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

The present disclosure further provides nanoparticle compositions comprise a lipid component including at least one compound according to Formulae IA, IB, IC, IIA, IIB, IIC, IID, IIE, IIF, including IAa-IAc, IBa-Ibe, ICa-ICc, IIAa-IIAb, IIBa, IICa, IIDa, IIEa-IIEb, and IIFa-IIFb, and any combination thereof. Nanoparticle compositions may also include a variety of other components. For example, the lipid component of a nanoparticle composition may include one or more other lipids in addition to a lipid according to Formula IA, IB, IC, IIA, IIB, IIC, IID, IIE, IIF, including IAa-IAc, IBa-Ibe, ICa-ICc, IIAa-IIAb, IIBa, IICa, IIDa, IIEa-IIEb, and IIFa-IIFb. Three-component Lipid Nanoparticle Compositions

The disclosure includes three-component LNP compositions containing:
1) a steroidal or structural lipid-containing component;
2) a PEGylated lipid-containing component; and
3) a cationic or ionizable lipid-containing component.

In some embodiments, the largest dimension of a nanoparticle composition is 1 µm or shorter (e.g., 1 µm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels.

### Cationic/Ionizable Lipid-Containing Component

A three component LNP composition of the present disclosure may include one or more cationic and/or ionizable lipids (e.g., lipids that may have a positive or partial positive charge at physiological pH) including, but not limited to, MC3, ALC-0315, ALC-0159, SM-102, DOTAP, Mol-111, Mol-114, MH-094, or a cationic and/or ionizable lipid disclosed in WO2017049245A2 (Benenato), lipids of Formulae IA, IB, IC, IIA, IIB, IIC, IID, IIE, IIF, including IAa-IAc, IBa-Ibe, ICa-ICc, IIAa-IIAb, IIBa, IICa, IIDa, IIEa-IIEb, and IIFa-IIFb, and any combination thereof.

### PEGylated Lipid-Containing Component

A three component LNP composition of the present disclosure may include one or more PEG or PEG-modified lipids. Such species may be alternately referred to as PEGylated lipids. A PEG lipid is a lipid modified with polyethylene glycol. A PEG lipid may be selected from the nonlimiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, or a mixture thereof. For example, a PEG lipid may be PEG-c-DOMG, PEG-DMG, PEG-DLPE, PEG-DMPE, PEG-DPPC, or a PEG-DSPE lipid.

### Steroidal/Structural Lipid-Containing Component

A three component LNP composition of the present disclosure may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, or a mixture thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

A three component LNP composition of the present disclosure is free of phospholipids. For example, the three component LNP composition of the present disclosure is free of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

### Adjuvants

In some embodiments, the three-component LNP may be combined in a composition with one or more adjuvants, e.g., Glucopyranosyl Lipid Adjuvant (GLA), CpG oligodeoxynucleotides (e.g., Class A or B), poly(I:C), aluminum hydroxide, Pam3CSK4, saponin extracts (e.g. Quil-A®), and Lipid A.

### Therapeutic Agents

Nanoparticle compositions comprising one or more lipids described herein may include one or more therapeutic and/or prophylactics. The disclosure features methods of delivering a therapeutic and/or prophylactic to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic.

The three-component LNP composition (also referred to herein as a “modified LNP” or a “mLNP”) may include one or more therapeutic and/or prophylactics. The disclosure features methods of delivering a therapeutic and/or prophylactic to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic.

Therapeutic and/or prophylactics include biologically active substances and are alternately referred to as “active agents.” A therapeutic and/or prophylactic may be a substance that, once delivered to a cell or organ, brings about a desirable change in the cell, organ, or other bodily tissue or system. Such species may be useful in the treatment of one or more diseases, disorders, or conditions. In some embodiments, a therapeutic and/or prophylactic is a small molecule drug useful in the treatment of a particular disease, disorder, or condition. Examples of drugs useful in the nanoparticle compositions include, but are not limited to, antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cystine arabinoside, anthracyclines, alkylative agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetolol), antihypertensive agents (e.g., clonidine and hydralazine), antidepressants (e.g., imipramine, amitriptyline, and doxepim), anti-conversants (e.g., phenytoin), antihistamines (e.g., diphenhydramine, chlorphenirimine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

### Polynucleotides and Nucleic Acids

In some embodiments, a therapeutic agent is a polynucleotide or nucleic acid (e.g., ribonucleic acid or deoxyribonucleic acid). The term “polynucleotide,” in its broadest sense, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In some embodiments, a therapeutic and/or prophylactic is an RNA. RNAs useful in the compositions and methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a mixture thereof. In certain embodiments, the RNA is an mRNA.

In certain embodiments, a therapeutic and/or prophylactic is an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. While exemplary polypeptides in the examples include polypeptides from respiratory syncytial virus (RSV) and Covid-19 as proof of concept, the lipids and compositions of the present disclosure are applicable to any mRNA molecules encoding any polypeptides of interest.

In other embodiments, a therapeutic and/or prophylactic is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a therapeutic and/or prophylactic is an shRNA or a vector or plasmid encoding the same. An shRNA may be produced inside a target cell upon delivery of an appropriate construct to the nucleus. Constructs and mechanisms relating to shRNA are well known in the relevant arts.

Nucleic acids and polynucleotides useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a nucleic acid or polynucleotide further includes a polyA region or a Kozak sequence (e.g., in the 5′-UTR). In some cases, polynucleotides may contain one or more intronic nucleotide sequences capable of being excised from the polynucleotide. In some embodiments, a polynucleotide or nucleic acid (e.g., an mRNA) may include a 5′ cap structure, a chain terminating nucleotide, a stem loop, a polyA sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′—O—methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine or 1-ethyl-pseudouridine), and/or a 5-substituted cytidine (e.g., 5-methyl-cytidine).

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a lipid according to Formulae IA, IB, IC, IIA, IIB, IIC, IID, IIE, IIF, including IAa-IAc, IBa-Ibe, ICa-ICc, IIAa-IIAb, IIBa, IICa, IIDa, IIEa-IIEb, and IIFa-IIFb, and any combination thereof, a phospholipid (such as an unsaturated lipid, e.g., DOPE or DSPC), a PEG lipid, and a structural lipid. The elements of the lipid component may be provided in specific fractions.

The three-component LNP composition may include, for example, the three components in the following relative mole percentages:
1) 5 to 60 mole% of a steroidal or structural lipid-containing component;
2) 0.5 to 20 mole% of a PEGylated lipid-containing component; and
3) 30 to 70 mole% of a cationic or ionizable lipid-containing component.

In one aspect, the three-component LNP composition contains the three components in the following relative mole percentages:
1) 20 to 50 mole% of a steroidal or structural lipid-containing component;
2) 0.8 to 10 mole% of a PEGylated lipid-containing component; and
3) 40 to 62 mole% of a cationic or ionizable lipid-containing component.

In one aspect, the three-component LNP composition contains the three components in the following relative mole percentages:
1) 25 to 46 mole% of a steroidal or structural lipid-containing component;
2) 1 to 7 mole% of a PEGylated lipid-containing component; and
3) 44 to 58 mole% of a cationic or ionizable lipid-containing component.

In one aspect, the three-component LNP composition contains the three components in the following relative mole percentages:
1) 35 to 44 mole% of a steroidal or structural lipid-containing component;
2) 1.2 to 5 mole% of a PEGylated lipid-containing component; and
3) 48 to 57 mole% of a cationic or ionizable lipid-containing component.

In one aspect, the three-component LNP composition contains the three components in the following relative mole percentages:
1) 37 to 43 mole% of a steroidal or structural lipid-containing component;
2) 1.4 to 3 mole% of a PEGylated lipid-containing component; and
3) 50 to 56 mole% of a cationic or ionizable lipid-containing component.

Any numerical value within the recited ranges and any combination of ranges and specific numerical values within the claimed ranges are contemplated and supported by the foregoing disclosures, i.e., 5 to 60 mole% of a steroidal or structural lipid-containing component includes any numerical value and range within the range of 5 to 60, e.g., 5, 5.01, 5.02, ...59.97, 59.98, 59.99, 60, 5-10, 5-20, 10-30, 15-25, etc. Similarly, 0.5 to 20 mole% of a PEGylated lipid-containing component includes any numerical value and range within the range of 0.5 to 20, e.g., 0.5, 0.501, 0.502, ...19.97, 19.98, 19.99, 20, 0.5-10, 0.52-15, 1-12, 5-13, etc. Similarly, 30 to 70 mole% of a cationic or ionizable lipid-containing component includes any numerical value and range within the range of 30 to 70, e.g., 30, 30.01, 30.02, ...69.97, 69.98, 69.99, 70, 30.5-68, 35-51, 40-52, 45-63, etc.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

### Pharmaceutical Compositions

Nanoparticle compositions may be formulated in whole or in part as pharmaceutical compositions. Pharmaceutical compositions may include one or more nanoparticle compositions. For example, a pharmaceutical composition may include one or more nanoparticle compositions including one or more different therapeutic and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington’s The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

In certain embodiments, the LNP is cryo-protected with 8% v/v sucrose. However, any cryo-protectant will suffice (e.g. trehalose). The concentration of the cryo-protectant can be from 4%-32% v/v.

In certain embodiments, the nanoparticle compositions and/or pharmaceutical compositions of the disclosure are refrigerated or frozen for storage and/or shipment (e.g., being stored at a temperature of 4° C. or lower, such as a temperature between about -150° C. and about 0° C. or between about -80° C. and about -20° C. (e.g., about -5° C., -10° C., -15° C., -20° C., -25° C., -30° C., -40° C., -50° C., -60° C., -70° C., -80° C., -90° C., -130° C. or -150° C.). In certain embodiments, the disclosure also relates to a method of increasing stability of the three-component LNP compositions and/or pharmaceutical compositions by storing the nanoparticle compositions and/or pharmaceutical compositions at a temperature of 4° C. or lower, such as a temperature between about -150° C. and about 0° C. or between about -80° C. and about -20° C., e.g., about -5° C., -10° C., -15° C., -20° C., -25° C., -30° C., -40° C., -50° C., -60° C., -70° C., -80° C., -90° C., -130° C. or -150° C.). For example, the three-component LNP compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and -20° C.). In one embodiment, the formulation is stabilized for at least 4 weeks at about 4° C. In certain embodiments, the pharmaceutical composition of the disclosure comprises a nanoparticle composition disclosed herein and a pharmaceutically acceptable carrier selected from one or more of Tris, an acetate (e.g., sodium acetate), an citrate (e.g., sodium citrate), saline, PBS, and sucrose. In certain embodiments, the carrier may be at a concentration of 1-100 mM (e.g., including but not limited to any numerical value or range within the range of 1-100 mM such as 1, 2, 3, 4, ...97, 98, 99, 100, 10-90 mM, 20-80 mM, 30-70 mM and so on).

LNP buffer exchange may be performed by dialysis, tangential flow filtration, or any other method that effectively removes and replaces buffer.

In certain embodiments, the pharmaceutical composition of the disclosure has a pH value between about 4 and 8 (e.g., 4, 4.1, 4.2, ... 6.8 6.9, 7.0, 7.1, 7.2, 7.3, 7.4, 7.5, 7.6, 7.7, 7.8, 7.9 or 8.0, or between 4 and 7 or between 5 and 6.5). For example, a pharmaceutical composition of the disclosure comprises a nanoparticle composition disclosed herein, Tris, saline and sucrose, and has a pH of about 7-8, which is suitable for storage and/or shipment at, for example, about -20° C. For example, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In certain embodiments, the pharmaceutical composition of the disclosure contain the therapeutic or prophylactic agent at a ratio of 0.05 to 25 mg/ml, 0.1 to 20 mg/ml, 0.2 to 18 mg/ml, 0.5 to 15 mg/ml, 0.7 to 12 mg/ml, 0.9 to 10 mg/ml, 1 to 8 mg/ml, 1.5 to 6 mg/ml, 2 to 5 mg/ml, 2.5 to 4 mg/ml, 0.5 to 3.0 mg/ml, 0.2 to 4.0 mg/ml, 0.4 to 2.0 mg/ml, and any numerical value or range within the range of 0.05 to 25 mg/ml.

Nanoparticle compositions and/or pharmaceutical compositions including one or more nanoparticle compositions may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including the three-component LNP compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer’s solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Methods of Producing Polypeptides in Cells

The present disclosure provides methods of producing a polypeptide of interest in a mammalian cell. Methods of producing polypeptides involve contacting a cell with a nanoparticle composition including an mRNA encoding the polypeptide of interest. Upon contacting the cell with the nanoparticle composition, the mRNA may be taken up and translated in the cell to produce the polypeptide of interest.

In general, the step of contacting a mammalian cell with a nanoparticle composition including an mRNA encoding a polypeptide of interest may be performed in vivo, ex vivo, in culture, or in vitro. The amount of nanoparticle composition contacted with a cell, and/or the amount of mRNA therein, may depend on the type of cell or tissue being contacted, the means of administration, the physiochemical characteristics of the nanoparticle composition and the mRNA (e.g., size, charge, and chemical composition) therein, and other factors. In general, an effective amount of the nanoparticle composition will allow for efficient polypeptide production in the cell. Metrics for efficiency may include polypeptide translation (indicated by polypeptide expression), level of mRNA degradation, and immune response indicators.

The step of contacting a nanoparticle composition including an mRNA with a cell may involve or cause transfection. Transfection may allow for the translation of the mRNA within the cell.

### Methods of Delivering Therapeutic Agents to Cells and Organs

The present disclosure provides methods of delivering a therapeutic and/or prophylactic to a mammalian cell or organ. Delivery of a therapeutic and/or prophylactic to a cell involves administering a nanoparticle composition including the therapeutic and/or prophylactic to a subject, where administration of the composition involves contacting the cell with the composition. For example, a protein, cytotoxic agent, radioactive ion, chemotherapeutic agent, or nucleic acid (such as an RNA, e.g., mRNA) may be delivered to a cell or organ. In the instance that a therapeutic and/or prophylactic is an mRNA, upon contacting a cell with the nanoparticle composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a nanoparticle composition may target a particular type or class of cells (e.g., cells of a particular organ or system thereof). For example, a nanoparticle composition including a therapeutic and/or prophylactic of interest may be specifically delivered to a mammalian liver, kidney, spleen, femur, or lung. Specific delivery to a particular class of cells, an organ, or a system or group thereof implies that a higher proportion of nanoparticle compositions including a therapeutic and/or prophylactic are delivered to the destination (e.g., tissue) of interest relative to other destinations, e.g., upon administration of a nanoparticle composition to a mammal. In some embodiments, specific delivery may result in a greater than 2 fold, 5 fold, 10 fold, 15 fold, or 20 fold increase in the amount of therapeutic and/or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, an ocular tissue (e.g., via intraocular, subretinal, or intravitreal injection), vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other therapeutic and/or prophylactics or elements (e.g., lipids or ligands) of a nanoparticle composition may be selected based on their affinity for particular receptors (e.g., low density lipoprotein receptors) such that a nanoparticle composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic (e.g., an mRNA) in a given dose, where a dose of 1 mg/kg (mpk) provides 1 mg of a therapeutic and/or prophylactic per 1 kg of subject body weight. In some embodiments, a dose of about 0.001 mg/kg to about 10 mg/kg of a therapeutic and/or prophylactic (e.g., mRNA) of a nanoparticle composition may be administered. In other embodiments, a dose of about 0.005 mg/kg to about 2.5 mg/kg of a therapeutic and/or prophylactic may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more therapeutic and/or prophylactics may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more nanoparticle compositions including one or more different therapeutic and/or prophylactics may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

### Example 1

A three-component LNP composition of the present disclosure was made containing 25 mM SM-102 in ethanol, 19 mM cholesterol in ethanol, and 0.75 mM DMG-PEG2000. Equal volumes of each lipid and of a blank ethanol were combined to give lipid mole ratios as follows: SM-102 (55.9%), cholesterol (42.4%), and DMG-PEG2000 (1.7%). The 0.13 mg/g mRNA in 25 mM sodium acetate pH 6.0 was prepared.

The LNP was formulated with a mRNA aqueous solution to lipid ethanolic solution ratio of 3:1 using microfludics to give unimodal peaks. The sample was then dialyzed using 10 kDa MWCO cassettes at 4° C. against 20 mM Tris-HCl, 8% sucrose to produce the final three-component LNP composition. The sample was concentrated to an mRNA concentration over 0.2 mg/mL by UV and filter-sterilization was performed. Surprisingly, the encapsulation was found to be comparable to current 4-component LNP systems.

A control (four-component LNP) composition was formulated using SM-102 (50%), cholesterol (38.5%), DSPC (10%), and DMG-PEG2000 (1.5%) in ethanol mixed by microfluidics with mRNA (0.13 mg/mL) in 25 mM sodium acetate pH 6.0 and dialyzed into 20 mM Tris-HCl pH 7.4, 8% sucrose (“RL-007”). The LNPs were filter-sterilized and concentrated to give an mRNA concentration over 0.2 mg/mL by UV.

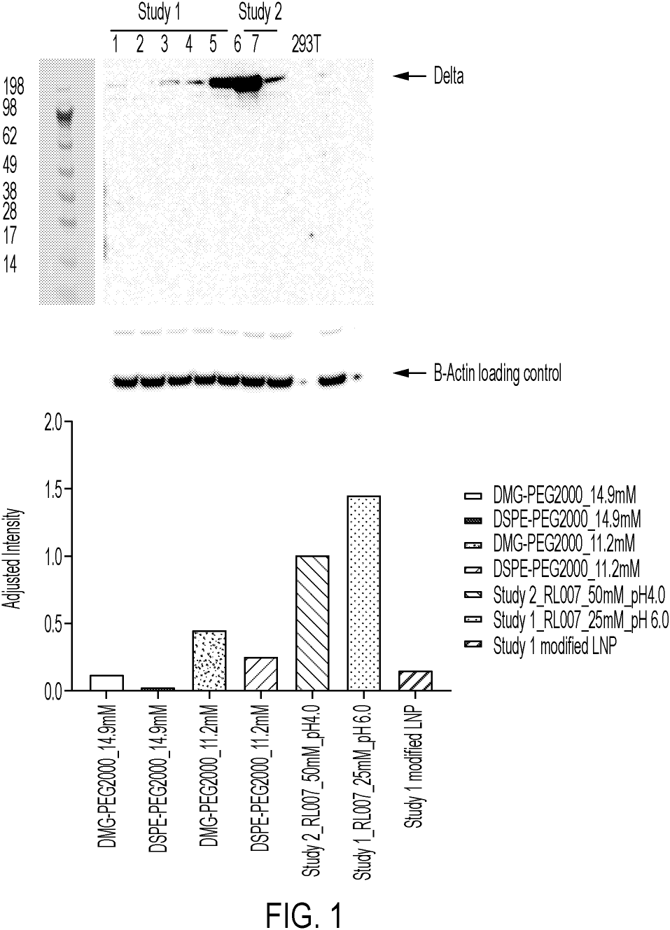

In vitro expression was determined and is shown in the western blot of FIG. 1, channels 6 (4-component control) and 7 (novel 3-component LNP) and in FIG. 1, lower the last (right side) two bars.

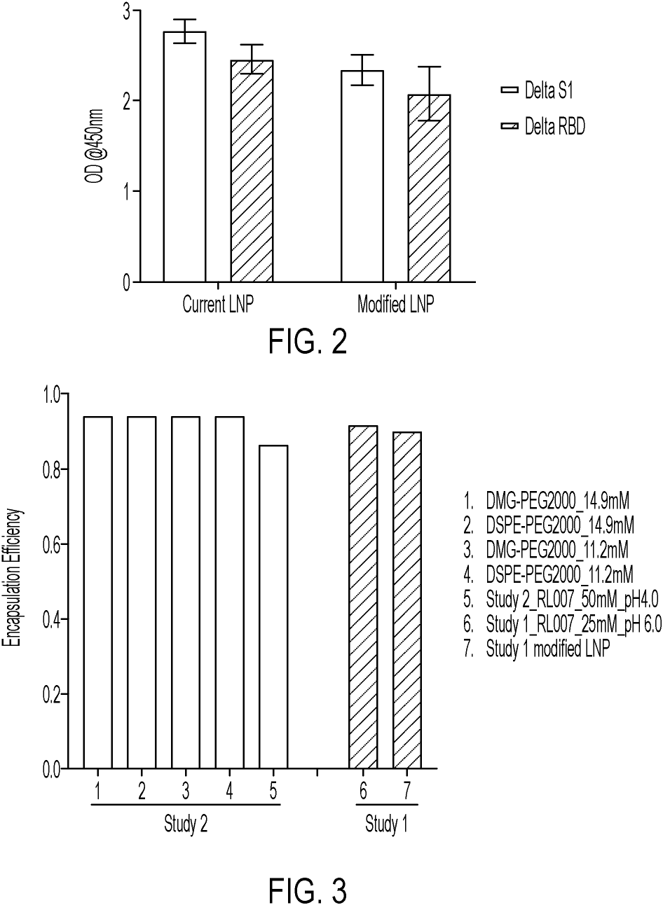

In vivo expression was determined from IM injection in mice. The expression after the first dose is shown in FIG. 2. In FIG. 2, the novel 3-component LNP composition was compared to a 4-component LNP control. The expression after one dose was very similar to the 4-component (DSPC-containing) control.

The encapsulation of both the 3-component LNP and the 4-component LNP control were excellent (FIG. 3, green).

### Example 2

Further formulations were made and tested as summarized in the following table and characterized in FIGS. 1-3.

| Composit ion Name | Cationic/Io nizable Componen t | PEGylated Component | Steroidal/Str uctural Component | Phospholipi d Component | Total Lipid Concentration | pH |
|---|---|---|---|---|---|---|
| Study 1 Modified LNP | 25 mM SM-102 (55.9 mole%) | 0.75 mM DMG-PEG2000 (1.7 mole%) | 19 mM cholesterol (42.4 mole%) | NONE | 11.2 mM | 6 |
| Study 1 Control -RL007 | 25 mM SM-102 (50.0 mole%) | 0.75 mM DMG-PEG2000 (1.5 mole%) | 19 mM cholesterol (38.5 mole%) | 5 mM DSPC (10 mole%) | 12.5 mM | 6 |
| Study 2 Modified LNP -DMG-PEG2000 - 14.9 mM | 25 mM SM-102 (55.9 mole%) | 0.75 mM DMG-PEG2000 (1.7 mole%) | 19 mM cholesterol (42.4 mole%) | NONE | 14.9 mM | 4 |
| Study 2 Modified LNP -DSPE-PEG2000 | 25 mM SM-102 (55.9 mole%) | 0.75 mM DMG-PEG2000 (1.7 mole%) | 19 mM cholesterol (42.4 mole%) | NONE | 14.9 mM | 4 |
| Study 2 Modified LNP -DMG-PEG2000 | 25 mM SM-102 (55.9 mole%) | 0.75 mM DMG-PEG2000 (1.7 mole%) | 19 mM cholesterol (42.4 mole%) | NONE | 11.2 mM | 4 |
| Study 2 Modified LNP -DSPE-PEG2000 | 25 mM SM-102 (55.9 mole%) | 0.75 mM DMG-PEG2000 (1.7 mole%) | 19 mM cholesterol (42.4 mole%) | NONE | 11.2 mM | 4 |
| Study 2 Control -RL-007 | 25 mM SM-102 (50.0 mole%) | 0.75 mM DMG-PEG2000 (1.5 mole%) | 19 mM cholesterol (38.5 mole%) | 5 mM DSPC (10 mole%) | 11.2 mM | 4 |

In another aspect of this invention, the same lipids were prepared in relation to the same 4-component control except, the formulation was performed with mRNA (0.13 mg/mL) in 50 mM sodium citrate, pH 4.0. The results are shown in FIG. 1, upper channel 3 and lower 3^{rd} bar with the control in channel 5 and bar 5. The encapsulation, determined by ribogreen assay, of the 3-component and 4-component systems were very similar.

In another aspect, the same components were prepared for LNP, but the total concentration of the lipids in ethanol solution was adjusted (see Table), see FIG. 1, channel 1 and bar 1. At the elevated concentration the in vitro data showed worse performance relative to the control used in the previous example. However, contrary to the in vitro data, the in vivo data show that the 3-component LNP of the present disclosure performed very well, which was very surprising.

In another aspect of this invention, a 3-component LNP was prepared using SM-102, cholesterol, and DSPE-PEG2000. The in vitro data were gathered, see FIG. 1 channel 4 and bar 4. In vitro expression was observed but it was less than the 3-component LNP using DMG-PEG2000 with same concentration and buffer conditions. The encapsulation was similar to the control (FIG. 3).

In another aspect of this invention, the previous 3-component LNP was prepared using lower concentreations of the lipids in ethanol, (FIG. 1, channel 2 and bar 2). The expression was the lowest of all of the attempted combinations of the three-component LNPs. The encapsulation was similar to the control (FIG. 3, bar 2).

### Example 3

In vivo expression of exemplary LNPs (mLNPs) and a 4-component LNP were tested in mice.

In each case, the Covid-19 Delta S1 or Delta RBD mRNA was prepared in a 50 mM sodium citrate buffer (pH 4) for RL007-pH4* and mLNP2-pH4. Or, mRNA was prepared in a 25 mM sodium acetate buffer (pH 4 for mLNP3-pH4, pH 5 for mLNP4-pH5, or pH 6 for mLNP1-pH6). The lipids for RL007-pH4* were prepared in stock solutions in ethanol: SM-102 (25 mM), DSPC (5 mM), cholesterol (19.3 mM), and DMG-PEG2000 (0.75 mM). Then, the lipids were mixed together in a 1:1:1:1 volume ratio to give a total lipid concentration of 12.5 mM. Each mLNP was prepared with the same stock solutions of SM-102 (25 mM), cholesterol (19.3 mM) and DMG-PEG2000 (0.75 mM). These three lipids were mixed in a 1:1:1 volume ratio and ethanol (200 proof) was added to dilute to same total volume as for RL007. The total lipid concentration for each mLNP is 11.2 mM.

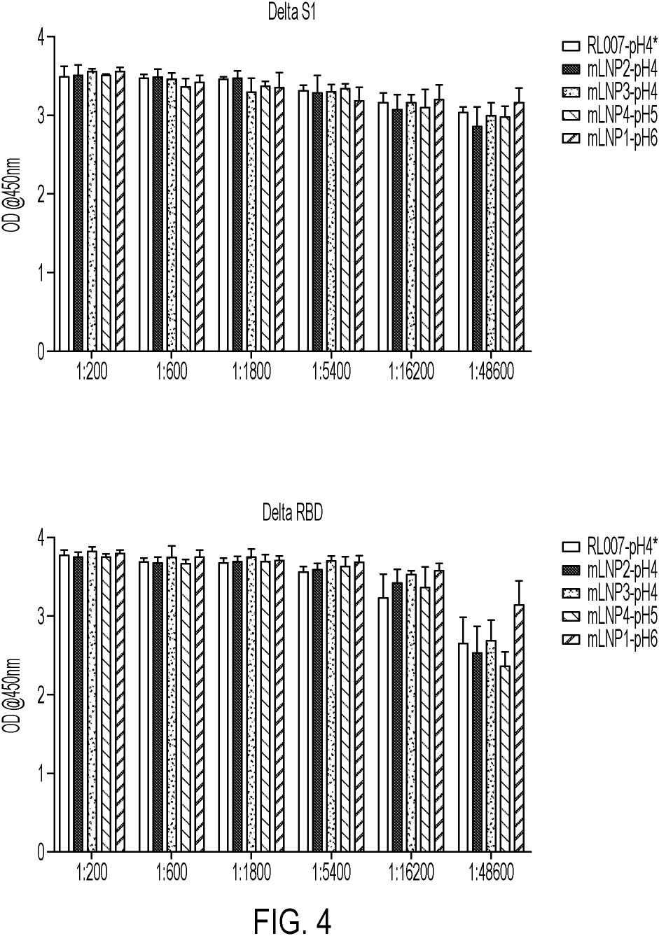

FIG. 4 shows plots that are in vivo expression of the mLNPs compared to the 4-component LNP. These data indicate that mLNPs performed as well as or somewhat better than 4-component LNPs after the second dose.

### Example 4

This example tested the in vitro expression comparison of 4-component LNPs with two ionizable lipids (SM-102 and Mol-111) with the same mRNA and relative lipid concentrations (SM-102_LNP and Mol-111_LNP) to 3-component LNPs with the same two ionizable lipids (SM-102 and Mol-111) with the same mRNA and relative lipid concentrations as the LNPs (SM-102_mLNP and Mol-111_mLNP).

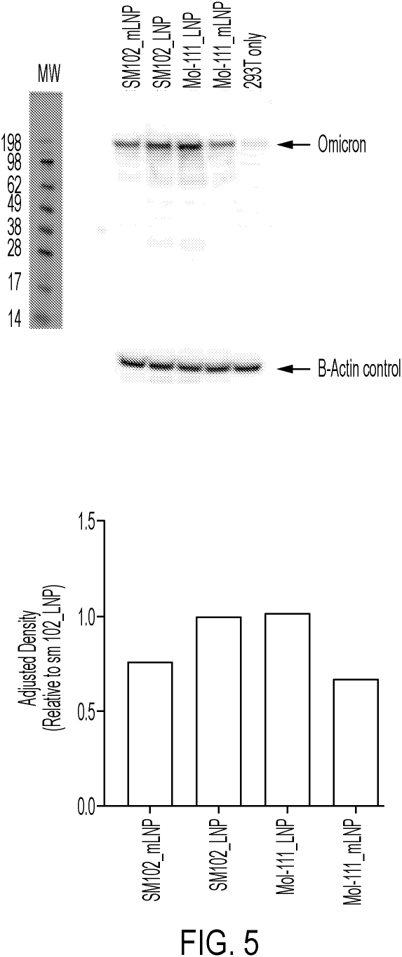

FIG. 5 shows in vitro western blot raw data and quantified results, which suggest that the mLNP was compatible with several types of lipids. In each case, the mRNA was a Covid Delta-Spike mRNA and was prepared at 0.13 mg/mL in 25 mM sodium acetate, pH 5.0. For the LNPs, the lipids were prepared as stock solutions using either SM-102 (25 mM) or Mol-111 (25 mM) with cholesterol (19.3 M), DSPC (5 mM), and DMG-PEG2000 (0.75 mM) mixed in a 1:1:1:1 volume ratio. For the mLNPs, the lipids were prepared as stock solutions and mixed to give final molar ratios for SM-102 or Mol-111 at 59.8 mol%, cholesterol (38.5 mol%) and DMG-PEG2000 (1.7 mol%). The data demostrate three component mLNPs for the inventive ionizable lipids that possess similar relative expression to four component LNPs.

### Example 5

This example tested in vivo expression across serial dilution demonstrating compatibility of exemplary LNPs (mLNPs) with mRNA of different lengths and multiple types of ionizable lipids.

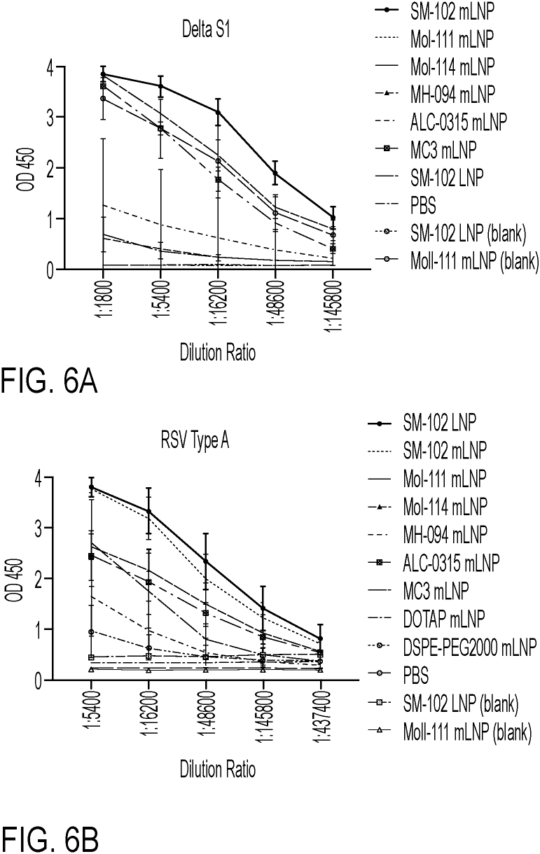

FIG. 6A shows the plot of mLNPs and LNP was either empty (SM-102 LNP (blank)) or contained Covid Delta Spike mRNA (~4000 nb), and FIG. 6B shows the plot for LNPs that contained RSV mRNA (~2000 nb).

Each plot contains multiple ionizable lipids (SM-102, Mol-11, Mol-114, MH-094, ALC-0315, and MC3). In each case, the concentration of the ionizable lipid in the stock solution was 25 mM and was mixed with the other lipid components to obtain a total lipid concentration of 12.5 mM for the LNP and 11.2 mM for the mLNP. For the RSV plot on the right, in addition to these lipids, the ionizable lipid was exchanged for a permanently cationic DOTAP or the DMG-PEG2000 was exchanged for DSPE-PEG2000. Both plots contain PBS and empty LNP controls using either SM-102 or Mol-111. These two plots show that mLNPs were effective delivery systems for longer mRNAs such as Covid and shorter mRNAs such as RSV; furthermore, mLNPs were compatible with a wide range of ionizable lipids for the delivery of either shorter or longer mRNAs.

### Example 6

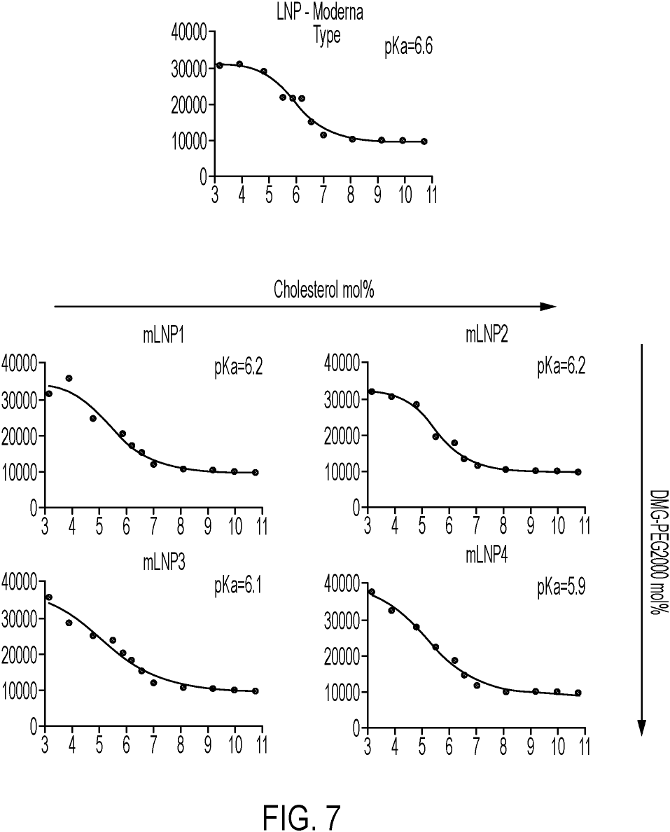

This example tested the difference in pKa between a control LNP and exemplary (mLNPs) as shown in FIG. 7.

The control LNP was prepared with RSV mRNA (0.13 mg/mL) in 25 mM sodium acetate (pH 5.0) with SM-102 (25 mM), cholesterol (19.3 mM), DSPC (5 mM), and DMG-PEG2000 (0.75 mM) stock solutions mixed in a 1:1:1:1 volume ratio and then formulated with the mRNA. The mLNP was prepared with RSV mRNA (0.13 mg/mL) in 25 mM sodium acetate (pH 5.0) formulated with combined stock solution of SM-102, cholesterol, and DMG-PEG2000 to give varied mole percentages of cholesterol and DMG-PEG2000. mLNP1 contains SM-102 (59.8 mol%), cholesterol (38.5 mol%), DMG-PEG2000 (1.7 mol%). mLNP2 contains SM-102 (59.3 mol%), cholesterol (40.0 mol%), DMG-PEG2000 (1.7 mol%). mLNP3 contains SM-102 (59.5 mol%), cholesterol (38.5 mol%), and DMG-PEG2000 (2.0 mol%). And mLNP4 contains SM-102 (58.0 mol%), cholesterol (40.0 mol%), and DMG-PEG2000 (2.0 mol%). As either cholesterol or DMG-PEG2000 increased with decreasing SM-102, pKa tended to decrease. The pKa data reveals that mLNP system is distinct and unique from the 4-component control LNP system in that the average mLNP pKa was lower than the average LNP pKa.

### Example 7

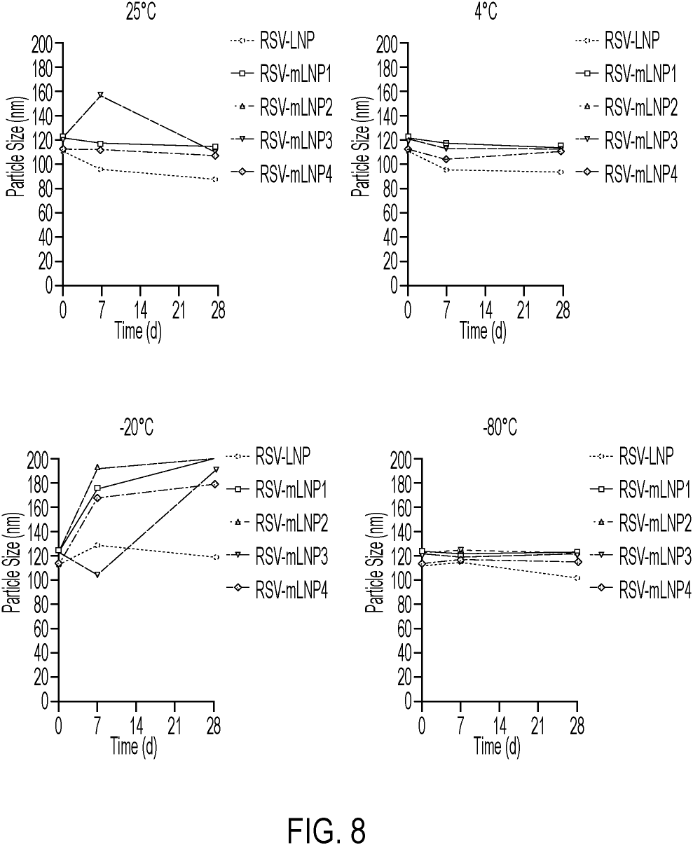

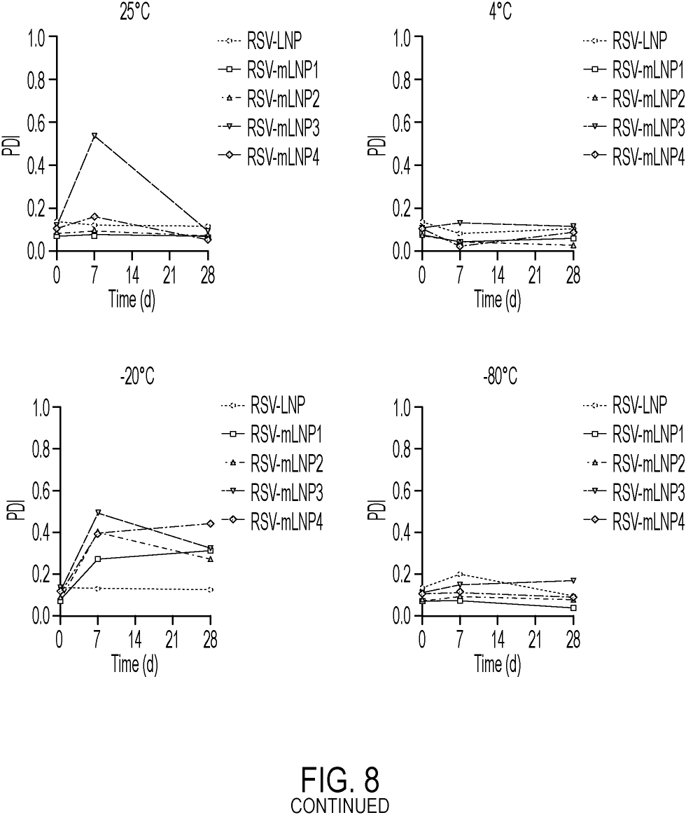

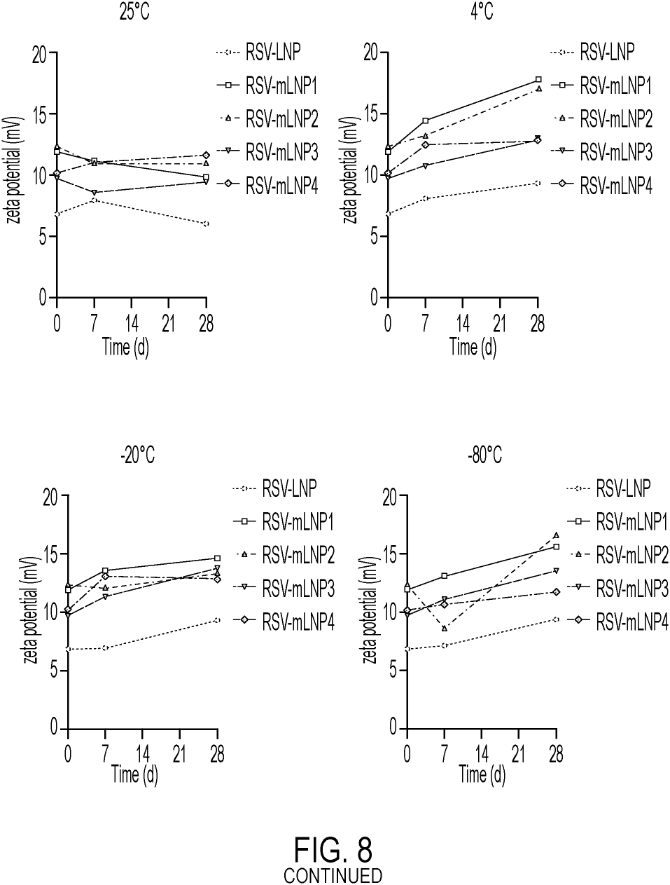

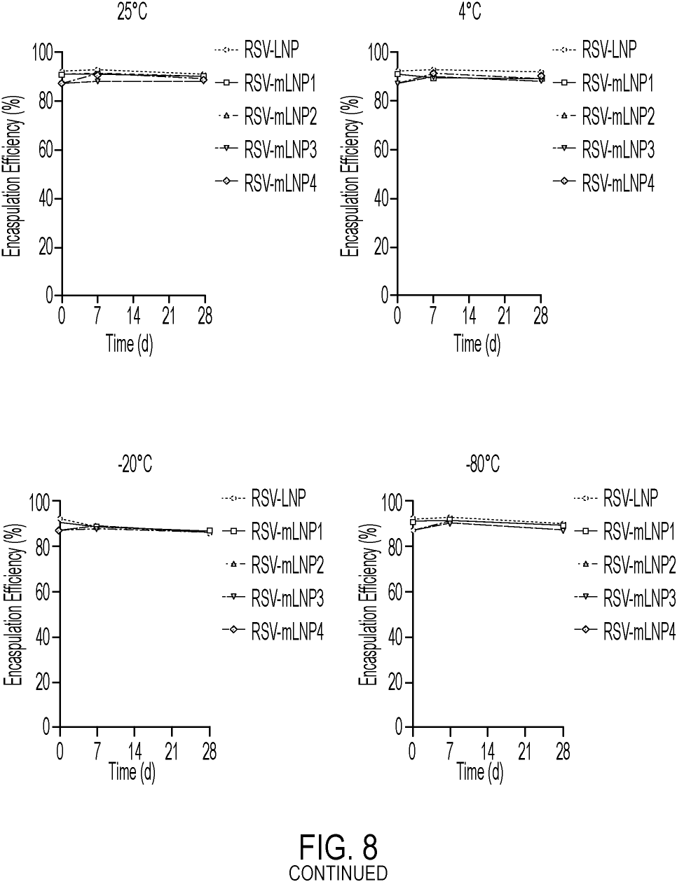

This example testesd stability of exemplary LNPs (mLNPs) compaed to a control LNP, as shown in FIG. 8.

In each case, the mRNA was RSV (2000 nb) prepared at 0.13 mg/mL in 25 mM sodium acetate and formulated with lipids SM-102 (25 mM), cholesterol (19.3 mM), DSPC (5 mM), and DMG-PEG2000 (0.75 mM) mixed in a 1:1:1:1 volume ratio. The mLNP was prepared with RSV mRNA (0.13 mg/mL) in 25 mM sodium acetate (pH 5.0) formulated with combined stock solution of SM-102, cholesterol, and DMG-PEG2000 to give varied mole percentages of cholesterol and DMG-PEG2000. mLNP1 contained SM-102 (59.8 mol%), cholesterol (38.5 mol%), DMG-PEG2000 (1.7 mol%). mLNP2 contained SM-102 (59.3 mol%), cholesterol (40.0 mol%), DMG-PEG2000 (1.7 mol%). mLNP3 contained SM-102 (59.5 mol%), cholesterol (38.5 mol%), and DMG-PEG2000 (2.0 mol%). And mLNP4 contained SM-102 (58.0 mol%), cholesterol (40.0 mol%), and DMG-PEG2000 (2.0 mol%). Stability was monitored by examining how size, polydispersity index (PDI), zeta potential and encapsulation efficiency vary at four different temperatures (25° C., 4° C., -20° C., and -80° C.). In both size and PDI, mLNP3 deviated from the LNP control. The remainder of the mLNPs behaved similarly to the LNP. The size, PDI, zeta potential, and encapsulation efficiency stability data indicates that mLNPs can be optimized to the same stability conditions as observed for LNPs in four metrics.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A three-component LNP composition wherein the three components are:
1) a steroidal or structural lipid-containing component;
2) a PEGylated lipid-containing component; and
3) a cationic or ionizable lipid-containing component.

2. The three-component LNP composition of claim 1, comprising the three components in the following relative mole percentages:
1) 5 to 60 mole% of a steroidal or structural lipid-containing component;
2) 0.5 to 20 mole% of a PEGylated lipid-containing component; and
3) 30 to 70 mole% of a cationic or ionizable lipid-containing component.

3. The three-component LNP composition of claim 1, comprising the three components in the following relative mole percentages:
1) 20 to 50 mole% of a steroidal or structural lipid-containing component;
2) 0.8 to 10 mole% of a PEGylated lipid-containing component; and
3) 40 to 62 mole% of a cationic or ionizable lipid-containing component.

4. The three-component LNP composition of claim 1, comprising the three components in the following relative mole percentages:
1) 25 to 46 mole% of a steroidal or structural lipid-containing component;
2) 1 to 7 mole% of a PEGylated lipid-containing component; and
3) 44 to 58 mole% of a cationic or ionizable lipid-containing component.

5. The three-component LNP composition of claim 1, comprising the three components in the following relative mole percentages:
1) 35 to 44 mole% of a steroidal or structural lipid-containing component;
2) 1.2 to 5 mole% of a PEGylated lipid-containing component; and
3) 48 to 57 mole% of a cationic or ionizable lipid-containing component.

6. The three-component LNP composition of claim 1, comprising the three components in the following relative mole percentages:
1) 37 to 43 mole% of a steroidal or structural lipid-containing component;
2) 1.4 to 3 mole% of a PEGylated lipid-containing component; and
3) 50 to 56 mole% of a cationic or ionizable lipid-containing component.

7. The three-component LNP composition of claim 1, wherein the cationic or ionizable lipid-containing component comprises MC3, ALC-0315, ALC-0159, SM-102, DOTAP, Mol-111, Mol-114, MH-094, a compound of Formula (IA), (IB), (IC), (IIA), (IIB), (IIC), (IID), (IIE), (IIF), or a combination thereof: 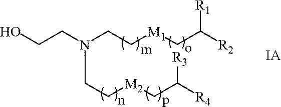
or a salt or isomer thereof, wherein
m is 0-9;
n is 0-9;
o is 0-12;
p is 0-12;
R₁ is a linear C₁₋₁₂ alkyl;
R₂ is H or a linear C₁₋₁₂ alkyl;
R₃ is a linear C₁₋₁₂ alkyl;
R₄ is H or linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; 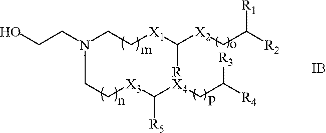
or a salt or isomer thereof, wherein
m is selected from 0-9;
n is selected from 0-9;
o is selected from 0-12;
p is selected from 0-12;
R is the side chain of an independently selected amino acid;
R₁ is a linear C₁₋₁₂ alkyl;
R₂ is H or linear C₁₋₁₂ alkyl;
R₃ is a linear C₁₋₁₂ alkyl;
R₄ is H or linear C₁₋₁₂ alkyl;
R₅ is the side chain of an independently selected amino acid;
X₁ is —OC(O)N(H)—, —C(O)N(H)—, —N(H)C(O)—, or —OC(O)—;
X₂ is —C(O)N(H)—, —C(O)O—, —N(H)C(O)—, or —N(H)C(O)—;
X₃ is —OC(O)N(H)—, —C(O)N(H)—, —N(H)C(O)—, or —OC(O)—; and
X₄ is —C(O)N(H)—, —C(O)O—, —N(H)C(O)—, or —N(H)C(O)—; 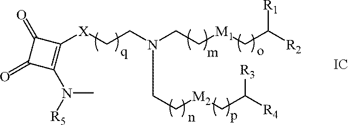
or a salt or isomer thereof, wherein
m is selected from 0-9;
n is selected from 0-9;
o is selected from 0-12;
p is selected from 0-12;
q is selected from 0-5;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl;
R₅ is H or CH₃;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; and
X is selected from —CH₂—, —O—, —S—, or —P(O)(OR)O—; 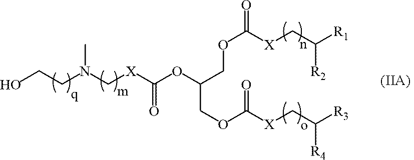
or a salt or isomer thereof, wherein
m is selected from 0-5;
n is selected from 0-12;
o is selected from 0-12;
q is selected from 1-3;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
X is selected from C(R)₂, N(R), or O, wherein R is independently selected from a methyl and H; 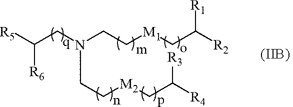
or a salt or isomer thereof, wherein
m is selected from 0-9;
n is selected from 0-9;
o is selected from 0-12;
p is selected from 0-12;
q is selected from 0-6;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; 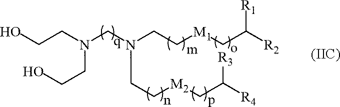
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; 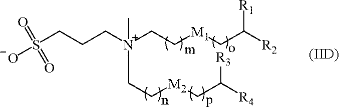
or a salt or isomer thereof, wherein
m is selected from 0-9;
n is selected from 1-7;
o is selected from 0-12;
p is selected from 0-12;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; 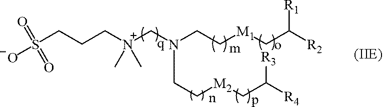
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)-, —C(O)S—, —SC(O)—, —OC(O)O—, —OC(O)N(R)—, or —N(R)C(O)O— groups, wherein R is independently selected from a methyl and H; 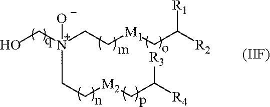
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

8. The three-component LNP composition of claim 1, wherein the steroidal or structural lipid-containing component is selected from the group consisting of cholesterol, fecosterol, sitosterol, ergosterol, campesterol, stigmasterol, brassicasterol, tomatidine, ursolic acid, alpha-tocopherol, or a mixture thereof; and or wherein the PEGylated lipid-containing component is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a mixture thereof.

9. A nanoparticle composition comprising the three-component LNP composition of claim 1, further comprising a therapeutic and/or prophylactic agent.

10. A compound selected from compounds of Formula IA, IB′, IB, IC, IIA, IIB, IIC, IID, IIE, IIF, where the compound of Formula IA is: 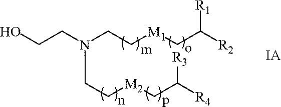
or a salt or isomer thereof, wherein
m is 0-9;
n is 0-9;
o is 0-12;
p is 0-12;
R₁ is a linear C₁₋₁₂ alkyl;
R₂ is H or a linear C₁₋₁₂ alkyl;
R₃ is a linear C₁₋₁₂ alkyl;
R₄ is H or linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H;
where the compound of Formula IB’ is: 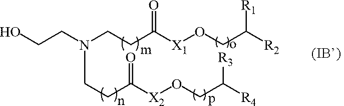
or a salt or isomer thereof, wherein
m is selected from 0-9;
n is selected from 0-9;
o is selected from 0-12;
p is selected from 0-12;
R₁ is a linear C₁₋₁₂ alkyl;
R₂ is H or a linear C₁₋₁₂ alkyl;
R₃ is a linear C₁₋₁₂ alkyl;
R₄ is H or a linear C₁₋₁₂ alkyl; and
X₁ and X₂ are independently selected from an amino acid, wherein the amino acid is Serine (S), Threonine (T), Cysteine (C), Selenocysteine (U), Glycine (G), Alanine (A), Isoleucine (I), Leucine (L), Methionine (M), or Valine (V);
where the compound of Formula IB is: 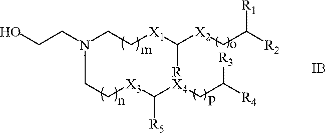
or a salt or isomer thereof, wherein
m is selected from 0-9;
n is selected from 0-9;
o is selected from 0-12;
p is selected from 0-12;
R is the side chain of an independently selected amino acid;
R₁ is a linear C₁₋₁₂ alkyl;
R₂ is H or linear C₁₋₁₂ alkyl;
R₃ is a linear C₁₋₁₂ alkyl;
R₄ is H or linear C₁₋₁₂ alkyl;
R₅ is the side chain of an independently selected amino acid;
X₁ is —OC(O)N(H)—, —C(O)N(H)—, —N(H)C(O)—, or —OC(O)—;
X₂ is —C(O)N(H)—, —C(O)O—, —N(H)C(O)—, or —N(H)C(O)—;
X₃ is —OC(O)N(H)—, —C(O)N(H)—, —N(H)C(O)—, or —OC(O)—; and
X₄ is —C(O)N(H)—, —C(O)O—, —N(H)C(O)—, or —N(H)C(O)—;
where the compound of Formula IC is: 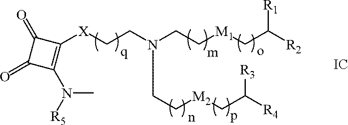
or a salt or isomer thereof, wherein
m is selected from 0-9;
n is selected from 0-9;
o is selected from 0-12;
p is selected from 0-12;
q is selected from 0-5;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is H or CH₃;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; and
X is selected from —CH₂—, —O—, —S—, or —P(O)(OR)O—;
where the compound of Formula IIA is: 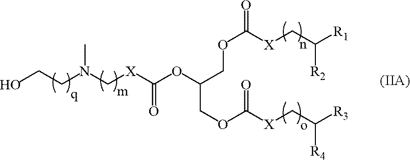
or a salt or isomer thereof, wherein
m is selected from 0-5;
n is selected from 0-12;
o is selected from 0-12;
q is selected from 1-3;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
X is selected from C(R)₂, N(R), or O, wherein R is independently selected from a methyl and H;
where the compound of Formula IIB is: 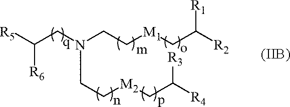
or a salt or isomer thereof, wherein
m is selected from 0-9;
n is selected from 0-9;
o is selected from 0-12;
p is selected from 0-12;
q is selected from 0-6;
R₁ is a linear C₁₋₁₂ alkyl;
R₂is H or a linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H;
where the compound of Formula IIC is: 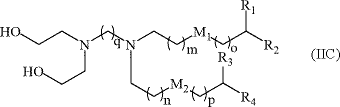
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl;
R₅ is a linear C₁₋₄ alkyl alcohol;
R₆ is a linear C₁₋₄ alkyl alcohol;
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H;
where the compound of Formula IID is: 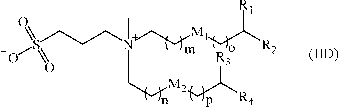
or a salt or isomer thereof, wherein
m is selected from 0-9;
n is selected from 1-7;
o is selected from 0-12;
p is selected from 0-12;
R₁ is a linear C₁₋₁₂ alkyl;
R₂is H or a linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H;
where the compound of Formula IIE is: 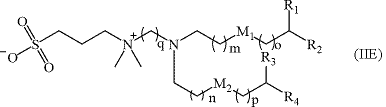
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂ is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H; or
where the compound of Formula IIF is: 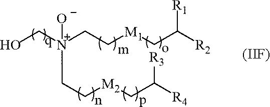
or a salt or isomer thereof, wherein
m is selected from 0-9;
n is selected from 0-9;
o is selected from 0-12;
p is selected from 0-12;
q is selected from 2-6;
R₁ is a linear C₁₋₁₂ alkyl;
R₂is H or linear C₁₋₁₂ alkyl;
R₃ is a linear C₁₋₁₂ alkyl;
R₄ is H or a linear C₁₋₁₂ alkyl; and
M₁ and M₂ are independently selected from —C(O)N(R)—, —N(R)C(O)—, —C(O)S—, —SC(O)—, —OC(O)O—, —OC(O)N(R)—, or —N(R)C(O)O— groups, wherein R is independently selected from a methyl and H.

11. The compound of claim 10, wherein the compound is: 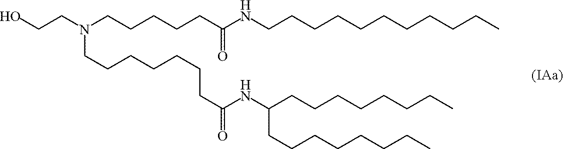 [SMILES: CCCCCCCCCCCNC(=O)CCCCCN(CCO)CCCCCCCC(=O)NC(CCCCCCCC)CCCCCCCC] 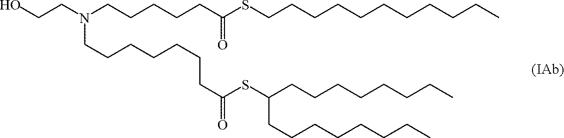 [SMILES: CCCCCCCCCCCSC(=O)CCCCCN(CCO)CCCCCCCC(=O)SC(CCCCCCCC)CCCCCCCC]
or 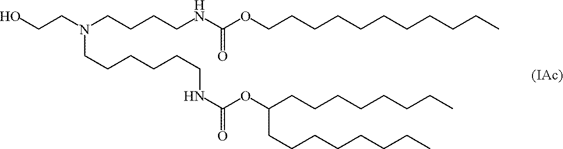 [SMILES: CCCCCCCCCCCOC(=O)NCCCCN(CCO)CCCCCCNC(=O)OC(CCCCCCCC)CCCCCCCC]
.

12. The compound of claim 10, wherein the compound is the compound of Formula IIB and R or R₅ comprises a side chain of a Serine (S), Threonine (T), Cysteine (C), Selenocysteine (U), Glycine (G), Alanine (A), Isoleucine (I), Leucine (L), Methionine (M), or Valine (V).

13. The compound of claim 10, wherein the compound is: 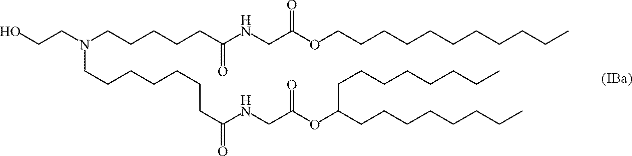 [SMILES: CCCCCCCCCCCOC(=O)CNC(=O)CCCCCN(CCO)CCCCCCCC(=O)NCC(=O)OC(CCCCCCCC)CCCCCCCC] 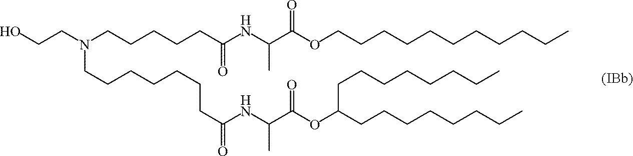 [SMILES: CCCCCCCCCCCOC(=O)C(C)NC(=O)CCCCCN(CCO)CCCCCCCC(=O)NC(C)C(=O)OC(CCCCCCCC)CCCCCCCC] 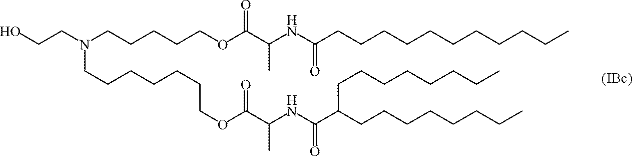 [SMILES: CCCCCCCCCCCC(=O)NC(C)C(=O)OCCCCCN(CCO)CCCCCCCOC(=O)C(C)NC(=O)C(CCCCCCCC)CCCCCCCC] 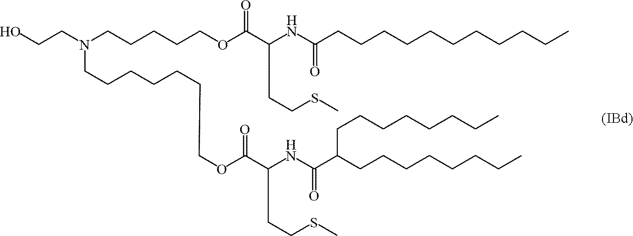 [SMILES: CCCCCCCCCCCC(=O)NC(CCSC)C(=O)OCCCCCN(CCO)CCCCCCCOC(=O)C(CCSC)NC(=O)C(CCCCCCCC)CCCCCCCC]
or 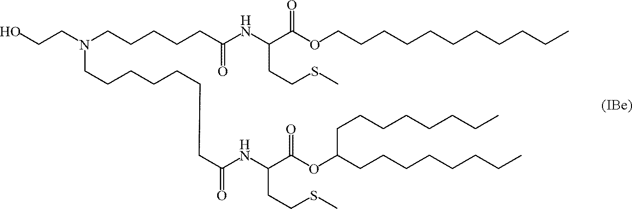 [SMILES: CCCCCCCCCCCOC(=O)C(CCSC)NC(=O)CCCCCN(CCO)CCCCCCCC(=O)NC(CCSC)C(=O)OC(CCCCCCCC)CCCCCCCC]
.

14. The compound of claim 10, wherein the compound is 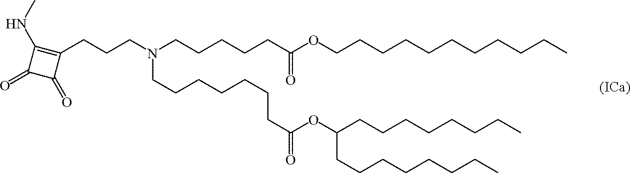 [SMILES: CCCCCCCCCCCOC(=O)CCCCCN(CCCCCCCC(=O)OC(CCCCCCCC)CCCCCCCC)CCCc1c(NC)c(=O)c1=O] 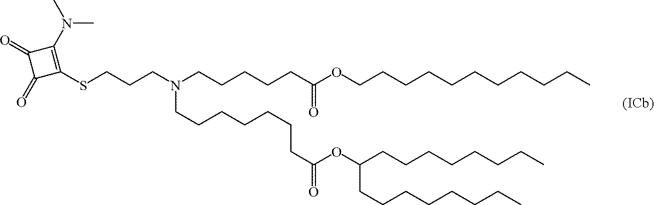 [SMILES: CCCCCCCCCCCOC(=O)CCCCCN(CCCCCCCC(=O)OC(CCCCCCCC)CCCCCCCC)CCCSc1c(N(C)C)c(=O)c1=O]
or 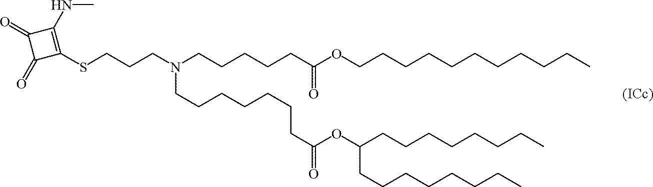 [SMILES: CCCCCCCCCCCOC(=O)CCCCCN(CCCCCCCC(=O)OC(CCCCCCCC)CCCCCCCC)CCCSc1c(NC)c(=O)c1=O]
.

15. The compound of claim 10, wherein the compound is: 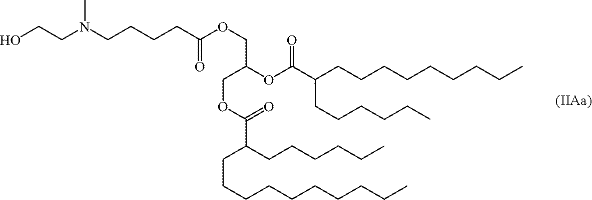 [SMILES: CCCCCCCCCCC(CCCCCC)C(=O)OCC(COC(=O)CCCCN(C)CCO)OC(=O)C(CCCCCC)CCCCCCCCC]
or 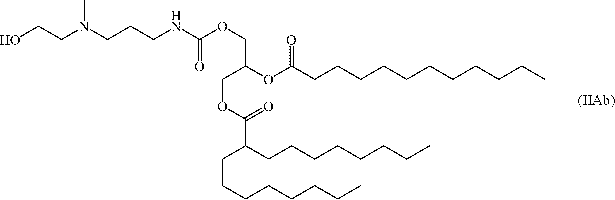 [SMILES: CCCCCCCCCCCC(=O)OC(COC(=O)NCCCN(C)CCO)COC(=O)C(CCCCCCCC)CCCCCCCC]
.

16. The compound of claim 10, wherein the compound is: 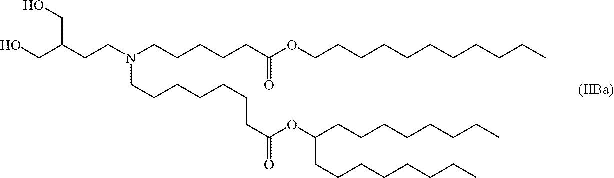 [SMILES: CCCCCCCCCCCOC(=O)CCCCCN(CCCCCCCC(=O)OC(CCCCCCCC)CCCCCCCC)CCC(CO)CO]
.

17. The compound of claim 10, wherein the compound is: 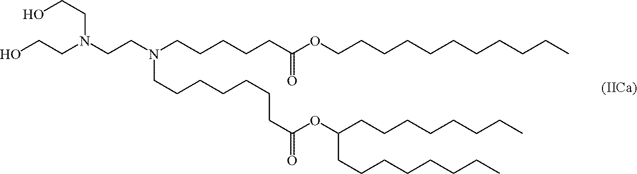 [SMILES: CCCCCCCCCCCOC(=O)CCCCCN(CCCCCCCC(=O)OC(CCCCCCCC)CCCCCCCC)CCN(CCO)CCO]
.

18. The compound of claim 10, wherein the compound is: 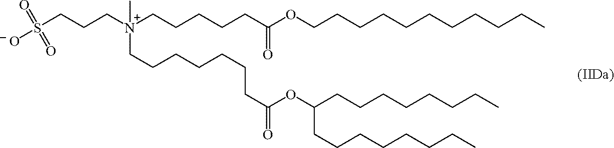 [SMILES: CCCCCCCCCCCOC(=O)CCCCC[N+](C)(CCCCCCCC(=O)OC(CCCCCCCC)CCCCCCCC)CCCS(=O)(=O)[O-]]
.

19. The compound of claim 10, wherein the compound is: 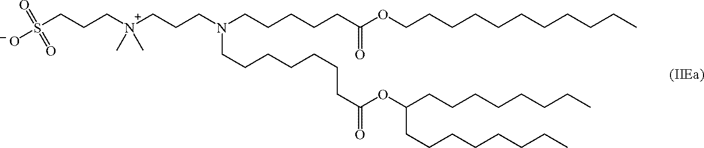 [SMILES: CCCCCCCCCCCOC(=O)CCCCCN(CCCCCCCC(=O)OC(CCCCCCCC)CCCCCCCC)CCC[N+](C)(C)CCCS(=O)(=O)[O-]]
or 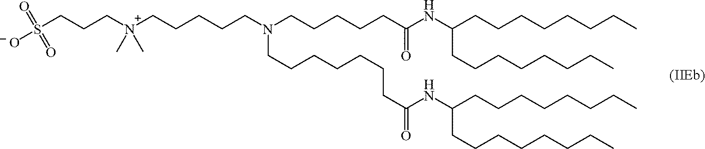 [SMILES: CCCCCCCCC(CCCCCCCC)NC(=O)CCCCCCCN(CCCCCC(=O)NC(CCCCCCCC)CCCCCCCC)CCCCC[N+](C)(C)CCCS(=O)(=O)[O-]]
.

20. The compound of claim 10, wherein the compound is: 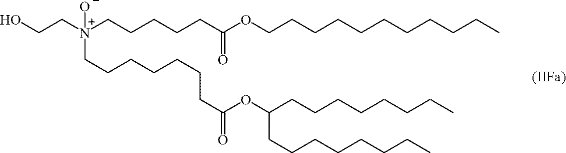 [SMILES: CCCCCCCCCCCOC(=O)CCCCC[N+]([O-])(CCO)CCCCCCCC(=O)OC(CCCCCCCC)CCCCCCCC]
or 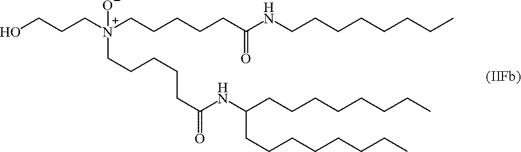 [SMILES: CCCCCCCCNC(=O)CCCCC[N+]([O-])(CCCO)CCCCCC(=O)NC(CCCCCCCC)CCCCCCCC]
.

21. A nanoparticle composition comprising a lipid component comprising the compound of claim 10.

22. The nanoparticle composition of claim 21, further comprising a therapeutic and/or prophylactic agent.

23. The nanoparticle composition of claim 21, further comprising a phospholipid.

24. The nanoparticle composition of claim 21, further comprising a structural lipid selected from the group consisting of cholesterol, fecosterol, sitosterol, ergosterol, campesterol, stigmasterol, brassicasterol, tomatidine, ursolic acid, alpha-tocopherol, or a mixture thereof.

25. The nanoparticle composition of claim 21, further comprising a PEG lipid selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or a mixture thereof.

26. The nanoparticle composition of claim 21, further comprising a cationic and/or ionizable lipid.

27. The nanoparticle composition of claim 21, wherein the nanoparticle composition: has an encapsulation efficiency of at least 80% when stored at 25° C., 4° C., -20° C., or -80° C. for at least 28 days; has a zeta potential of 5-15 mV when stored at 25° C., 4° C., -20° C., or -80° C. for at least 28 days; has a PDI of less than 0.2 when stored at 25° C., 4° C., -20° C., or -80° C. for at least 28 days; and/or has a particle size of less than 140 nm when stored at 25° C., 4° C., -20° C., or -80° C. for at least 28 days.

28. A method of delivering a therapeutic and/or prophylactic agent to a cell, the method comprising administering to a subject the nanoparticle composition of claim 22, said administering comprising contacting the cell with the nanoparticle composition, whereby the therapeutic and/or prophylactic agent is delivered to the cell.

29. A pharmaceutical composition comprising the nanoparticle composition of claim 22 and a pharmaceutically acceptable carrier.

30. A method of delivering a therapeutic and/or prophylactic agent to a cell of a subject, the method comprising administering the pharmaceutical composition of claim 29 to the subject.
